(19) United States
(12) Patent Application Publication
(10) Pub. No.: US 2015/0125871 A1
(43) Pub. Date: May 7, 2015

(54) METHODS FOR DECONTAMINATING CIRCUITS FOR PRODUCING GLUCOSE POLYMERS AND HYDROLYSATES OF GLUCOSE POLYMERS

(71) Applicants: Sophie Duvet, Quesnoy Sur Deule (FR); Hela Hacine-Guerbi, Villeneuve D'Ascq (FR); Pierre Lanos, La Bassee (FR); Fabrice Allain, Lille (FR); Mathieu Carpentier, SAINT Andre Lez Lille (FR); Agnes Denys, Lille (FR)

(72) Inventors: Sophie Duvet, Quesnoy Sur Deule (FR); Hela Hacine-Guerbi, Villeneuve D'Ascq (FR); Pierre Lanos, La Bassee (FR); Fabrice Allain, Lille (FR); Mathieu Carpentier, SAINT Andre Lez Lille (FR); Agnes Denys, Lille (FR)

(73) Assignee: ROQUETTE FRERES, LESTREM (FR)

(21) Appl. No.: 14/403,600

(22) PCT Filed: May 28, 2013
(86) PCT No.: PCT/FR2013/051181

§ 371 Date: Nov. 25, 2014

(30) Foreign Application Priority Data
May 29, 2012 (FR) 1254935
Jul. 16, 2012 (FR) 1256848
Oct. 18, 2012 (FR) 1259923
Jan. 16, 2013 (FR) 1350353
Mar. 27, 2013 (FR) 1352748

(51) Int. Cl. G01N 33/50; G01N 33/68; C12Q 1/68; C08B 37/00

(52) U.S. Cl. CPC G01N 33/5055; C08B 37/0009; G01N 33/6863; C12Q 1/6897; G01N 2333/70596
USPC 435/6.13; 536/123.1; 435/7.24

(57) ABSTRACT

The present invention concerns a method for determining the impact of a production step or a purification step on the presence or nature of pro-inflammatory contaminating molecules in glucose polymers or the hydrolysates of same by using an in vitro test of inflammatory response using cell lines. It further concerns an optimised method of producing or purifying glucose polymers or the hydrolysates of same comprising an analysis of the pro-inflammatory contaminating molecules in glucose polymers or the hydrolysates of same and the selection of production or purification steps optimised with respect to the presence and nature of the pro-inflammatory contaminating molecules.

The present invention relates to methods for decontaminating circuits for producing or purifying glucose polymers, more particularly those intended for the food sectors (fiber-rich health ingredients) and medical fields (peritoneal dialysis), or hydrolysates of glucose polymers, more particularly those intended for the medical fields (injectable non-pyrogenic glucose).

### TECHNICAL BACKGROUND OF THE INVENTION

The applicant company has chosen to develop its invention in a field which is known for the dangerousness of the contaminants of microbial origin capable of developing in circuits for producing glucose polymers or in those for producing hydrolysates thereof, said contaminants being responsible for possible:
food poisoning,
inflammatory reactions very harmful to human health.

In the context of a food safety approach, as in that of a health safety approach, it is therefore important to be sure of the absence of contaminants of microbial origin, both in the form of living cells and in the form of cell debris, by all appropriate technical means, in particular:
the definition of safe production circuits, by setting up appropriate purification devices and techniques,
the definition of methods for effective identification and assaying of contaminants.

For example, in the case of peritoneal dialysis, a certain number of ingredients must be prepared under the strictest conditions of purity.

Peritoneal dialysis is in fact a type of dialysis of which the objective is to remove waste such as urea, creatinine, excess potassium or surplus water that the kidneys do not or no longer manage to purify from the blood plasma. This medical treatment is indicated in the case of terminal chronic renal failure.

The dialysates most commonly used are composed of a buffer solution (of lactate or of bicarbonate) at acidic pH (5.2-5.5) or physiological pH (7.4) to which are added electrolytes (sodium, calcium, magnesium, chlorine) and especially an osmotic agent (glucose or a glucose polymer, such as “icodextrin” present in the ambulatory peritoneal dialysis solution EXTRANEAL® sold by the company BAXTER).

The glucose polymer, such as icodextrin mentioned above, is preferred to glucose as osmotic agent since, because of its small size, glucose, which rapidly crosses the peritoneum, leads to a loss of osmotic gradient in the 2 to 4 hours of infusion.

In the more particular field of the use of glucose polymers for continuous ambulatory peritoneal dialysis, it very quickly became apparent that these starch hydrolysates (mixture of glucose, and of glucose oligomers and polymers) could not be used as such.

European patent application EP 207 676 teaches that glucose polymers forming clear and colorless solutions at 10% in water, having a weight-average molecular weight (Mw) of 5000 to 100 000 daltons and a number-average molecular weight (Mn) of less than 8000 daltons are preferred.

Such glucose polymers also preferably comprise at least 80% of glucose polymers of which the molecular weight is between 5000 and 50 000 daltons, little or no glucose or glucose polymers with a DP less than or equal to 3 (molecular weight 504) and little or no glucose polymers with a molecular weight greater than 100 000 (DP of about 600).

In other words, the preferred glucose polymers are glucose polymers with a low polydispersity index (value obtained by calculating the Mw/Mn ratio).

The methods proposed in said patent application EP 207 676 for obtaining these glucose polymers with a low polydispersity index from starch hydrolysates consist:
either in carrying out a fractional precipitation of a maltodextrin with a water-miscible solvent,
or in carrying out a molecular filtration of this same maltodextrin through various membranes possessing an appropriate cut-off or exclusion threshold.

In the two cases, these methods are aimed at removing both the very high-molecular-weight polymers and the low-molecular-weight monomers or oligomers.

However, these methods do not provide satisfaction both from the point of view of their implementation and from the point of view of the yields and the quality of the products that they make it possible to obtain.

In the interests of developing a method for producing a completely water-soluble glucose polymer with a low polydispersity index preferentially less than 2.5, preferably having an Mn of less than 8000 daltons and having an Mw of between 12 000 and 20 000 daltons, said method lacking the drawbacks of the prior art, the applicant company endeavored to solve this problem in its patent EP 667 356, by starting from a hydrolyzed starch rather than from a maltodextrin.

The glucose polymer obtained by chromatographic fractionation then preferably contains less than 3% of glucose and of glucose polymers having a DP of less than or equal to 3 and less than 0.5% of glucose polymers having a DP of more than 600.

It is finally henceforth accepted by the experts in the field of peritoneal dialysis that these glucose polymers, used for their osmotic power, are entirely satisfactory.

However, risks of microbial contamination of these preparations intended for peritoneal dialysis are to be deplored.

It is in fact known that circuits for producing glucose polymers can be contaminated with microorganisms, or with pro-inflammatory substances contained in said microorganisms.

The contamination of corn or wheat starches by microorganisms of yeast, mold and bacteria type, and more particularly by acidothermophilic bacteria of Alicyclobacillus acidocaldarius type (extremophilic bacteria which grow in the hot and acidic zones of the circuit) is, for example, described in the starch industry.

The major risk for the patient who receives these contaminated products is then peritonitis.

These episodes of peritonitis are caused by intraperitoneal bacterial infections, and the diagnosis is usually easily established through positive dialysate cultures.

“Sterile peritonitis”, which is described as aseptic, chemical or culture-negative peritonitis, is, for its part, typically caused by a chemical irritant or a foreign body.

Since the introduction of icodextrin for the preparation of peritoneal dialysis solutions, isolated cases of aseptic peritonitis have been reported, that can be linked to various causes, and in particular induction by pro-inflammatory substances potentially present.

Aseptic inflammatory episodes are therefore major complications observed after injections of dialysis solutions.

While some of these inflammatory episodes are linked to a problem of chemical nature (accidental injection of chemical contaminants or incorrect doses of certain compounds), the majority of cases are directly associated with the presence of contaminants of microbial origin that are present in the solutions used to prepare the dialysis solutions.

Lipopolysaccharides (LPSs) and peptidoglycans (PGNs) are the main contaminants of microbial origin which present a high risk of triggering an inflammation even when they are present in trace amounts.

It is, moreover, to the applicant company's credit to have also taken into account the presence of molecules capable of exacerbating the inflammatory response induced by these contaminants, such as PGN depolymerization products, the minimum structure of which that is still bioactive is muramyl dipeptide (MDP).

These derivatives, considered in isolation, are not very inflammatory in vitro and give a significant response for values greater than 1 μg/ml.

In addition to the PGN depolymerization products, formylated microbial peptides, the prototype of which is f-MLP (formyl-Met-Leu-Phe tripeptide), also have a substantial synergistic activity. Originally, these peptides were identified for their chemoattractant activity on leukocytes, although they are incapable of inducing a cytokine response per se.

It is therefore important not to neglect these “small molecules”, since they can indirectly account for aseptic inflammatory episodes by exacerbating the effects of traces of PGN and/or of LPS.

The pharmacopeia proposes a battery of tests for detecting pyrogenic substances:
the test for detecting bacterial endotoxins, majority components of Gram-negative bacteria (LAL test),
the rabbit pyrogen test.

Although generally reliable, these two tests have their limits. The rabbit pyrogen test is based on the indirect detection of pyrogenic substances by measuring an elevation in the temperature of the rabbit that is being injected with the product containing these substances (febrile response).

This test can produce false negatives, if the undesirable substance has a biological activity that is too weak or a concentration that is too low to induce a systemic pyrogenic response.

Moreover, this substance may have a biological activity or concentration sufficient to produce a local inflammatory reaction.

The LAL test, for its part, detects only bacterial endotoxins (LPS) and also β-glucans, which are components of the walls of fungal flora. The other biological impurities (DNA, peptidoglycans, etc) are not detected.

The manifestation of aseptic peritonitis observed with the peritoneal dialysis solutions containing icodextrin therefore, for certain cases, attest to the way that some substances can escape the tests described in the pharmacopeia and can be responsible for undesirable clinical effects.

In order to remedy this situation, the company BAXTER had proposed making efforts to detect the Gram-positive microbial contaminants.

In particular, in its patent EP 1 720 999, the company BAXTER proposes developing a method based on the detection of peptidoglycans, which are the major components of Gram-positive bacterial membranes, in particular in glucose polymers for the preparation of peritoneal dialysis solutions.

In other words, in order to prevent the occurrence of these episodes of aseptic peritonitis, the company BAXTER proposed, for the production and use of peritoneal dialysis solutions, a protocol for detecting peptidoglycans in the peritoneal dialysis solution.

Moreover, while the upstream treatment of glucose polymers was mentioned in this patent EP 1 720 999, this treatment is only by means of affinity resins capable of trapping the peptidoglycans as such.

It was not therefore envisioned to modify the method for producing glucose polymers in such a way that the final product is free of contamination by acidothermophilic bacteria of Alicyclobacillus acidocaldarius type or by membrane debris of these particular bacteria.

On the other hand, in its international patent application WO 2010/125315, the applicant company provided, by means of a safe preparation and purification method, substances for peritoneal dialysis of better quality, in the case in point glucose polymers, in order to ensure that these substances are effectively free of contaminating substances.

The applicant company has implemented a noteworthy purification method, combining a certain number of steps of treatment with activated carbon/granular black carbon, of filtration (microfiltration and ultrafiltration) and of heat treatment organized in a manner suitable for preventing any contamination.

However, this method can be improved, and the applicant company has devoted itself to developing detection and assaying methods which are more effective than those accessible in the prior art, in order to better define the key method steps to be implemented in order to ensure optimum safety of production lines, in particular of glucose polymers.

From all the aforementioned, there remains an unsatisfied need to provide, by means of a safe preparation and purification method, substances for therapeutic applications of better quality, in the case in point polymers of glucose and hydrolysates thereof, in order to ensure that these substances are effectively free of contaminants.

The applicant company has therefore found that this need can be satisfied by implementing the appropriate purification steps, the effectiveness of which can be measured by means of methods for detecting and assaying contaminants which are entirely specific.

Over the past few years, numerous tests using primary cells have been developed in order to replace animal models in inflammatory response tests.

However, these in vitro models are subject to considerable interindividual variability, which can be responsible for experimental biases.

Conversely, monocyte cell lines give constant responses, thereby explaining why the tests currently undergoing development increasingly use cells of this type in culture. However, these tests have the drawback of giving an overall inflammatory response to all the contaminants present as a mixture in a solution, and, consequently, do not make it possible to characterize the nature of the contaminant.

It is also important to note that the exacerbated inflammatory response is visible for cytokines of the acute phase of the inflammation, such as TNF-α (Tumor Necrosis Factor alpha), IL-1β (interleukin 1β) and chemokines such as CCL5 (Chemokine (C—C unit) ligand 5)/RANTES (Regulated upon Activation, Normal T-cell Expressed, and Secreted), but not or barely for IL-6 (interleukin 6).

Thus, the methods based on the production of the latter (US 2009/0239819 and US 2007/0184496) are not suitable for detecting contaminants as a mixture in a solution.

The applicant company has come to the following conclusions:
(i) it is difficult to detect bacterial contaminants present in trace amounts in biological solutions,
(ii) it is important not to be limited to the detection of PGNs and of LPSs, owing to the synergistic effects,
(iii) it is necessary to develop new detection methods which are sensitive and reproducible, and
(iv) it is advantageous to use sensitive and reproducible detection methods capable of characterizing the nature of the contaminants.

It has therefore been to the applicant company's credit to have developed sensitive and effective methods for detecting microbial contaminants which have a pro-inflammatory action, below the threshold of sensitivity of the procedures currently used and/or described in the literature, and subsequently to have identified the family, or even the nature, of the pro-inflammatory molecules present in trace amounts in batches originating from production circuits.

### SUMMARY OF THE INVENTION

The present invention relates to a method for testing the effect of a production step or production steps or the effectiveness of a purification step or purification steps on the presence or the nature of pro-inflammatory molecules in glucose polymers or hydrolysates thereof, comprising:
a) providing glucose polymers or hydrolysates thereof;
b) optionally, detecting or assaying the pro-inflammatory molecules in the glucose polymers or hydrolysates thereof provided in step a);
c) carrying out the production or purification step or steps on the glucose polymers or hydrolysates thereof provided in step a);
d) detecting or assaying the pro-inflammatory molecules in the glucose polymers or hydrolysates thereof obtained after step c);
e) determining the effectiveness or the impact of step c) on the presence or the nature of the pro-inflammatory molecules;
in which the step for detecting or assaying the pro-inflammatory molecules in the glucose polymers or hydrolysates thereof comprises an in vitro inflammatory response test using a cell line, the cell line being either a macrophage or a macrophage-differentiated cell line, or a cell expressing one or more TLR (Toll Like Receptor) or NOD (Nucleotide-binding Oligomerization Domain-containing protein) receptors, such as TLR2, TLR4 or NOD2, and making it possible to detect the responses of the receptor or receptors, or a combination thereof.

It also relates to an optimized method for producing or purifying glucose polymers or hydrolysates thereof, comprising:
a) providing glucose polymers or hydrolysates thereof;
b) detecting or assaying the pro-inflammatory molecules in the glucose polymers or hydrolysates thereof provided in step a);
c) selecting the step or steps for producing or purifying the glucose polymers or hydrolysates thereof that is or are suitable for the pro-inflammatory molecules present in the glucose polymers or hydrolysates thereof;
d) optionally, carrying out the selected production or purification step or steps on the glucose polymers or hydrolysates thereof provided in step a); and
e) optionally, detecting or assaying the pro-inflammatory molecules in the glucose polymers or hydrolysates thereof obtained after step d);
in which the step for detecting or assaying the pro-inflammatory molecules in the glucose polymers or hydrolysates thereof comprises an in vitro inflammatory response test using a cell line, the cell line being either a macrophage or a macrophage-differentiated cell line, or a cell expressing one or more TLR (Toll Like Receptor) or NOD (Nucleotide-binding Oligomerization Domain-containing protein) receptors, such as TLR2, TLR4 or NOD2, and making it possible to detect the responses of the receptor or receptors, or a combination thereof.

In a first aspect, the in vitro inflammatory response test may comprise bringing the glucose polymers or hydrolysates thereof into contact with the MDP- or LPS-sensitized, macrophage-differentiated THP-1 cell line, the pro-inflammatory molecules being detected or assayed by measuring the amount of RANTES or TNF-a produced by the cell line.

In a second aspect, the in vitro inflammatory response test may comprise bringing the glucose polymers or hydrolysates thereof into contact with a macrophage line transfected with a reporter gene, the transcription of which is under the direct control of the inflammatory signaling pathways, such as the Raw-Blue™ line, the pro-inflammatory molecules being detected or assayed by measuring the activity or the signal of the reporter gene.

In a third aspect, the in vitro inflammatory response test may comprise bringing the glucose polymers or hydrolysates thereof into contact with a cell line expressing the TLR2 receptor and a reporter gene, the transcription of which is under the direct control of the TLR2 signaling pathways, such as the HEK-Blue™ hTLR2 line, the pro-inflammatory molecules being detected or assayed by measuring the activity or the signal of the reporter gene.

In a fourth aspect, the in vitro inflammatory response test may comprise bringing the glucose polymers or hydrolysates thereof into contact with a cell line expressing the NOD2 receptor and a reporter gene, the transcription of which is under the direct control of the NOD2 signaling pathways, such as the HEK-Blue™ hNOD2 line, the pro-inflammatory molecules being detected or assayed by measuring the activity or the signal of the reporter gene.

In a fifth aspect, the in vitro inflammatory response test may comprise bringing the glucose polymers or hydrolysates thereof into contact with a cell line expressing the TLR4 receptor and a reporter gene, the transcription of which is under the direct control of the TLR4 signaling pathways, such as the HEK-Blue™ hTLR4 line, the pro-inflammatory molecules being detected or assayed by measuring the activity or the signal of the reporter gene.

In a sixth aspect, the in vitro inflammatory response test may comprise bringing the glucose polymers or hydrolysates thereof into contact with:
a. the MDP- or LPS-sensitized, macrophage-differentiated THP-1 cell line, the pro-inflammatory molecules being detected or assayed by measuring the amount of RANTES or TNF-α produced by the cell line; and/or
b. a macrophage line transfected with a reporter gene, the transcription of which is under the direct control of the inflammatory signaling pathways, such as the Raw-Blue™ line, the pro-inflammatory molecules being detected or assayed by measuring the activity or the signal of the reporter gene; and/or
c. a cell line expressing the TLR2 receptor and a reporter gene, the transcription of which is under the direct control of the TLR2 signaling pathways, such as the HEK-Blue™ hTLR2 line, the pro-inflammatory molecules being detected or assayed by measuring the activity or the signal of the reporter gene; and/or
d. a cell line expressing the NOD2 receptor and a reporter gene, the transcription of which is under the direct control of the NOD2 signaling pathways, such as the HEK-Blue™ hNOD2 line, the pro-inflammatory molecules being detected or assayed by measuring the activity or the signal of the reporter gene; and/or
e. a cell line expressing the TLR4 receptor and a reporter gene, the transcription of which is under the direct control of the TLR4 signaling pathways, such as the HEK-Blue™ hTLR4 line, the pro-inflammatory molecules being detected or assayed by measuring the activity or the signal of the reporter gene; and/or
f. a control line not transfected with an immunity receptor.

In a seventh aspect, the in vitro inflammatory response test may comprise bringing the glucose polymers or hydrolysates thereof into contact with:
a. a macrophage line transfected with a reporter gene, the transcription of which is under the direct control of the inflammatory signaling pathways, such as the Raw-Blue™ line, the pro-inflammatory molecules being detected or assayed by measuring the activity or the signal of the reporter gene;
b. a cell line expressing the TLR2 receptor and a reporter gene, the transcription of which is under the direct control of the TLR2 signaling pathways, such as the HEK-Blue™ hTLR2 line, the pro-inflammatory molecules being detected or assayed by measuring the activity or the signal of the reporter gene; and/or
c. a cell line expressing the TLR4 receptor and a reporter gene, the transcription of which is under the direct control of the TLR4 signaling pathways, such as the HEK-Blue™ hTLR4 line, the pro-inflammatory molecules being detected or assayed by measuring the activity or the signal of the reporter gene; and/or
d. a cell line expressing the NOD2 receptor and a reporter gene, the transcription of which is under the direct control of the NOD2 signaling pathways, such as the HEK-Blue™ hNOD2 line, the pro-inflammatory molecules being detected or assayed by measuring the activity or the signal of the reporter gene; and
e. a control line not transfected with an immunity receptor, such as the HEK-Blue™ Null2 line.

Preferably, the pro-inflammatory molecules are molecules of bacterial origin, preferably selected from PGNs, LPSs, lipopeptides, PGN depolymerization products, in particular MDP, formylated microbial peptides such as f-MLP, and β-glucans.

Preferably, the production or purification step or steps is or are chosen from steps of heat treatment, of acidification, of passing over activated carbon, of passing over adsorption resins, of ultrafiltration, of filtration, or of chemical or enzymatic hydrolysis.

Preferably, the glucose polymers are selected from icodextrin and maltodextrins, in particular branched or unbranched maltodextrins, and the glucose polymer hydrolysates are a product of total hydrolysis, such as dextrose monohydrate.

The samples of glucose polymers or of hydrolysates thereof are prefiltered, in particular with a cut-off threshold at 30 kDa, and the filtrate is brought into contact with the cell line used in the test.

### DETAILED DESCRIPTION OF THE INVENTION

The present invention therefore relates to a method for testing the impact or the effect of a production step or production steps or the effectiveness of a purification step or purification steps on the presence or the nature of pro-inflammatory molecules in glucose polymers or hydrolysates thereof, comprising:
a) providing glucose polymers or hydrolysates thereof;
b) optionally, detecting or assaying the pro-inflammatory molecules in the glucose polymers or hydrolysates thereof provided in step a);
c) carrying out the production or purification step or steps on the glucose polymers or hydrolysates thereof provided in step a);
d) detecting or assaying the pro-inflammatory molecules in the glucose polymers or hydrolysates thereof obtained after step c);
e) determining the effectiveness or the impact of step c) on the presence or the nature of the pro-inflammatory molecules;
in which the step for detecting or assaying the pro-inflammatory molecules in the glucose polymers or hydrolysates thereof comprises an in vitro inflammatory response test using a cell line, the cell line being either a macrophage or a macrophage-differentiated cell line, or a cell expressing one or more TLR (Toll Like Receptor) or NOD (Nucleotide-binding Oligomerization Domain-containing protein) receptors, such as TLR2, TLR4 or NOD2, and making it possible to detect the responses of the receptor or receptors, or a combination thereof.

The method may comprise, in particular in the context of step e), a comparison of the pro-inflammatory molecules in the glucose polymers or hydrolysates thereof, detected or assayed in steps b) and d). Thus, a decrease in the amount of the pro-inflammatory molecules or of some of these molecules is indicative of the effectiveness of a production or purification step for the decontamination of the glucose polymers or hydrolysates thereof. The amount and the nature of the pro-inflammatory molecules will be determined by the methods described in detail below.

The objective of this method is in particular the development of an optimized method for decontaminating glucose polymers or hydrolysates thereof, in particular glucose polymers for the preparation of a peritoneal dialysis solution, this method preferably comprising detecting or assaying the pro-inflammatory molecules by an in vitro inflammatory response test.

In particular, once the impact or the effectiveness of the purification or production steps on the pro-inflammatory molecules has been characterized, in particular according to their presence and their nature, those skilled in the art are capable of choosing the steps most suitable considering the pro-inflammatory molecules present in the glucose polymers or hydrolysates thereof. Thus, the optimized method comprises:
a) providing glucose polymers or hydrolysates thereof;
b) detecting or assaying the pro-inflammatory molecules in the glucose polymers or hydrolysates thereof provided in step a);
c) selecting the step or steps for producing or purifying the glucose polymers or hydrolysates thereof that is or are suitable for the pro-inflammatory molecules present in the glucose polymers or hydrolysates thereof;
d) optionally, carrying out the selected production or purification step or steps on the glucose polymers or hydrolysates thereof provided in step a); and
e) optionally, detecting or assaying the pro-inflammatory molecules in the glucose polymers or hydrolysates thereof obtained after step d);
in which the step for detecting or assaying the pro-inflammatory molecules in the glucose polymers or hydrolysates thereof comprises an in vitro inflammatory response test using a cell line, the cell line being either a macrophage or a macrophage-differentiated cell line, or a cell expressing one or more TLR (Toll Like Receptor) or NOD (Nucleotide-binding Oligomerization Domain-containing protein) receptors, such as TLR2, TLR4 or NOD2, and making it possible to detect the responses of the receptor or receptors, or a combination thereof.

The glucose polymers or hydrolysates thereof may be for peritoneal dialysis, enteral and parenteral feeding, and the feeding of newborns.

In one preferred embodiment, the glucose polymers, which will be prepared in the context of the present invention, are icodextrin or maltodextrins (branched or unbranched, as will be described hereinafter).

The glucose polymer hydrolysates to which reference is made here are taken to be in particular the product of total hydrolysis, such as non-pyrogenic dextrose monohydrate, sold under the brand LYCADEX® PF by the applicant company.

They can be decontaminated at one or more stages of their preparation, and in particular at the level of the raw material, at any step in their method of preparation, and/or at the level of the final product of the method.

Thus, the glucose polymers or hydrolysates thereof provided in the methods according to the present invention correspond to the raw material, to the product at any level of the preparation method or to the final product.

The pro-inflammatory contaminants are especially molecules of bacterial origin. They may be in particular PGNs, LPSs, lipopeptides, PGN depolymerization products, in particular MDP, formylated microbial peptides such as f-MLP, β-glucans, etc.

The methods for measuring the in vitro inflammatory responses which are used in the context of the present invention in order to monitor the effectiveness of the decontamination steps of the methods for preparing glucose polymers for therapeutic use in humans (e.g. peritoneal dialysis solutions) are based on cell tests (“bio-assays”) using lines of monocyte/macrophage type (THP-1, and/or Raw-Blue™) and transfected lines expressing a specific receptor of natural immunity (HEK-Blue™)

The THP-1 line (88081201, ECACC) is a human promonocyte line. For the pro-inflammatory response tests, the cells are differentiated into monocytes/macrophages for 3 days in the presence of phorbol ester (PMA).

For the tests carried out according to the present invention, the macrophages or macrophage-differentiated cells, in particular the macrophage-differentiated THP-1 cells, are sensitized in the presence of MDP, in particular S. aureus MDP. This is because MDP is a weak inflammatory inducer, but it is known to act in synergy with other inflammatory molecules. This property is based on the fact that these molecules act via the intervention of receptors other than the MDP receptor, essentially TLRs. Consequently, the presence of MDP will exacerbate the inflammatory response induced by the contaminants present in the solutions of glucose polymers or of hydrolysates thereof, thus making it possible to detect low doses of contaminants. Preferably, the MDP is added to the sample at a concentration of more than 1 μg/ml, preferably at a concentration of between 1 and 100 μg/ml. In one quite particularly preferred embodiment, the MDP is added to the sample at a concentration of 10 μg/ml.

Alternatively, the macrophages or macrophage-differentiated cells, in particular the macrophage-differentiated THP-1 cells, can be sensitized in the presence of molecules other than MDP. Indeed, LPS, in particular an LPS from E. coli, can also be used. It can be added to the sample at a concentration of at least 10 pg/ml, for example at a concentration of 25 pg/ml.

In one preferred embodiment, the macrophages or macrophage-differentiated cells, in particular the macrophage-differentiated THP-1 cells, are used at a density of between 0.5 and 1×10⁶ cells/ml of culture medium, preferably between 0.7 and 0.8×10⁶ cells/ml, and even more preferably approximately 0.75×10⁶ cells/ml.

The in vitro inflammatory response test is based on the measurement of RANTES production by sensitized THP-1 cells. This is because the prior studies showed that the assay of this chemokine is suitable for detecting low doses of contaminants, in particular endotoxins, in glucose polymer solutions. Alternatively, the in vitro inflammatory response test can also be based on the measurement of TNF-α production by sensitized THP-1 cells. The assaying of the cytokines can be carried out by any means well known to those skilled in the art, and in particular by ELISA. In one preferred embodiment, the test comprises the measurement of TNF-α production after 8 h of stimulation. In another preferred embodiment, the test comprises the measurement of RANTES production after 20 h of stimulation, in particular by means of an ELISA assay.

This first test makes it possible to detect in particular the contamination of glucose polymers or of hydrolysates thereof with PGNs and/or LPSs, preferably with PGNs of medium size (in particular approximately 120 kDa) and/or LPSs, even more particularly with LPSs.

The Raw-Blue™ line is a line of mouse macrophages transfected with a reporter gene producing a secreted form of alkaline phosphatase (SEAP: secreted embryonic alkaline phosphatase), the transcription of which is under the direct control of the inflammatory signaling pathways. The advantage of this line is that it naturally expresses virtually all the innate immunity receptors, including the TLR2, TLR4 and NOD2 receptors. Thus, these cells will respond to the majority of inflammatory contaminants, and the response will be monitored by measuring the enzymatic activity of the SEAP produced. Preferably, this line is used in the test at a cell density of approximately 0.5×10⁶ cells/well. The bringing of the preparation of glucose polymers or of hydrolysates thereof into contact with the cells lasts approximately 16 to 24 h.

The cell lines, in particular HEK-Blue™ (InvivoGen), are lines modified by stable transfection with a vector encoding an innate immunity receptor, in particular human (h) receptors. They are also cotransfected with the reporter gene, in particular a reporter gene producing SEAP, the synthesis of which is under the direct control of the signaling pathway associated with the receptor overexpressed. Preferably, this reporter gene encodes a colored or fluorescent protein or a protein of which the activity can be measured with or without substrate. The detection of the activity of the signal of the reporter gene indicates that the sample contains contaminants capable of activating one or more innate immunity receptors and of triggering an inflammatory reaction. The use of these lines makes it possible to target certain families of molecules of microbial origin according to the receptor expressed. Preferably, cell lines expressing either hTLR2 or hTLR4 or hNOD2 are used. In addition, a control line which expresses no innate immunity receptor is also used. The use of this control line is of use for verifying that the solutions of glucose polymers or of hydrolysates thereof do not induce the production of the reporter gene via a parasitic mechanism, such as a toxicity mechanism.

For the tests according to the present invention, four lines are preferably used:
HEK-Blue™ hTLR2 line: this line expressing the hTLR2 receptor responds specifically to TLR2 agonists (PGNs and lipopeptides especially). Its use therefore makes it possible to know the level of these contaminants in the triggering of the inflammatory responses,
HEK-Blue™ hTLR4 line: this line expressing the hTLR4 receptor responds specifically to LPSs. Its use therefore makes it possible to know the level of these contaminants in the triggering of the inflammatory responses,
HEK-Blue™ hNOD2 line: this line expressing the hNOD2 receptor responds specifically to NOD2 agonists. Its use therefore makes it possible to know the level of MDP and related molecules in the triggering of the inflammatory responses,
HEK-Blue™ Null2 line: it is a control line, not transfected with an immunity receptor. Its use is necessary in order to verify that the solutions of glucose polymers or of hydrolysates thereof do not induce SEAP production via a toxicity mechanism.

However, it should be noted that those skilled in the art can also use other commercial lines (Imgenex) or they can prepare lines.

In one preferred embodiment, the cell lines are used at a density between 0.5 and 1×10⁶ cells/ml of culture medium, and the bringing of the preparation of glucose polymers or hydrolysates thereof into contact with the cells lasts approximately 16 to 24 h.

A quantification for contaminants can be carried out by means of a dose-response curve. This dose-response curve can in particular be produced with the same cells, under the same conditions, with increasing doses of contaminants. The dose-response curves are in particular produced with LPS, PGN, lipopeptide, β-glucan and MDP standards. Preferably, such a dose-response curve can be produced for cells expressing TLR4 (for example, THP-1, HEK-Blue™ hTLR4 and Raw-Blue™) with increasing doses of LPS, for cells expressing TLR2 (for example, THP-1, HEK-Blue™ hTLR2 and Raw-Blue™) with increasing doses of PGN, and for cells that are reactive via NOD2 (for example, HEK-Blue™ hNOD2) with increasing doses of MDP.

In particular embodiments, the THP-1, Raw-Blue™ and HEK-Blue™ lines are incubated with increasing concentrations of standards, and the cell response is measured by quantifying RANTES production by ELISA for the THP-1 line and measurement of the reporter gene, in particular of the enzymatic activity of SEAP for the Raw-Blue™ and HEK-Blue™ lines.

The test according to the invention makes it possible to identify the contaminant or contaminants capable of triggering an inflammatory reaction. Thus, the line expressing NOD2, in particular HEK-Blue™ hNOD2, makes it possible quite particularly to detect a contamination with PGN depolymerization products and MDP, preferably MDP. The line expressing TLR2, in particular HEK-Blue™ hTLR2 and/or Raw-Blue™, makes it possible quite particularly to detect a contamination with PGNs. Moreover, the macrophages, in particular the THP-1 macrophages, and the line expressing TLR4, in particular HEK-Blue™ hTLR4, make it possible quite particularly to detect a contamination with LPSs.

In one preferred embodiment, the in vitro inflammatory response test includes tests with the following cell lines:
macrophages, in particular THP-1 macrophages, a cell line which makes it possible to detect the activity of a TLR2 receptor, in particular the HEK-Blue™ hTLR2 line, a cell line which makes it possible to detect the activity of a TLR4 receptor, in particular the HEK-Blue™ hTLR4 line, a cell line which makes it possible to detect the activity of a NOD2 receptor, in particular the HEK-Blue™ hNOD2 line, and, optionally but preferably, a control line, in particular the HEK-Blue™ Null2 line; or
a line of macrophages transfected with a reporter gene, in particular the Raw-Blue™ line, a cell line which makes it possible to detect the activity of a TLR2 receptor, in particular the HEK-Blue™ hTLR2 line, a cell line which makes it possible to detect the activity of a TLR4 receptor, in particular the HEK-Blue™ hTLR4 line, a cell line which makes it possible to detect the activity of a NOD2 receptor, in particular the HEK-Blue™ hNOD2 line, and, optionally but preferably, a control line, in particular the HEK-Blue™ Null2 line.

In the preferred embodiment, the presence of contaminants in the various samples is tested using the five cell types presented above, so as to have an idea of the general inflammatory response, and also of the responses specific to certain contaminants:
MDP-sensitized THP—I line: any contaminants with strong reactivity for LPSs,
Raw-Blue™ line: any contaminants with strong reactivity for PGNs,
HEK-Blue™ hTLR2 line: PGNs and other TLR2 ligands (lipopeptides, etc),
HEK-Blue™ hTLR4 line: LPS,
HEK-Blue™ hNOD2 line: MDP and PGN depolymerization products,
HEK-Blue™ Null2 line: negative control.

In the method according to the present invention, the production or purification step or steps can be chosen from steps of heat treatment, of acidification, of passing over activated carbon, of passing over adsorption resins, of ultrafiltration, of filtration, or of chemical or enzymatic hydrolysis, or combinations thereof. Various parameters can be tested for each step, making it possible to select those which are the most effective. For example, in the case of passing over activated carbon, various qualities of activated carbon and combinations thereof can be tested. In the case of an ultrafiltration, it will be possible to test various cut-off thresholds and/or to combine them. In the case of a heat treatment, it will be possible to vary the treatment temperature and time. In the case of an enzymatic treatment, it will be possible to vary the enzyme or enzymes used, their concentration and their treatment conditions.

In one very particular embodiment, the treatments are carried out on samples prepared at 32% (weight/volume) in non-pyrogenic water (for injection), and then the solutions are filtered through 0.22 μm. For the cell tests, the samples are diluted to 1/10 in the cell culture medium (final concentration: 3.2% (w/v)).

The samples of glucose polymers or hydrolysates thereof can in addition be subjected to enzymatic or chemical treatments or to filtration steps prior to the test for detecting or assaying the pro-inflammatory molecules. Optionally, the results obtained before and after these treatment or filtration steps can be compared.

Thus, a sample of glucose polymers or hydrolysates thereof can be treated with a mutanolysin prior to the test. This enzyme, by virtue of its muramidase activity, is capable of depolymerizing PGNs. For example, the enzyme at a concentration of approximately 2500 U/ml can be placed in the presence of the sample, optionally diluted so as to have a glucose polymer concentration of 7.5 to 37.5% (weight/volume), for 6 to 16 h, preferably approximately 16 h. The sample thus treated will then be subjected to the test with one or more cell line or lines according to the present invention.

Alternatively, the sample of the preparation of glucose polymers or of hydrolysates thereof can be filtered prior to the test. The purpose of this filtration is essentially to remove the high-molecular-weight molecules, such as the high-molecular-weight PGNs, and to carry out the test on the filtrate in order to analyze quite particularly the contaminants of small sizes. The cut-off threshold for the filtration can, for example, be between 30 kD and 150 kD, preferably between 30 and 100 kD or between 30 and 50 kD, and in particular approximately 30 kD. In one preferred embodiment, the cell tests are carried out on the fractions obtained by ultrafiltration, in particular with cut-off thresholds of 30 and 100 kDa. Preferably, the filtration is carried out by ultrafiltration. It can also be carried out by any means known to those skilled in the art. Thus, the sample having been thus filtered, the filtrate will be subjected to the cell tests according to the present invention. The comparison of the results obtained without or before filtration will make it possible to deduce the specific inflammatory contribution of the molecules of small sizes. Moreover, this makes it possible to verify whether the production or purification steps modify the size of the contaminants (hydrolysis compared with aggregation), and/or do not remove certain contaminants of defined size.

Moreover, treatment of the samples with lysozyme and/or β-glucanase makes it possible to remove the PGN and/or the β-glucans, and to thus know the significance of the other TLR2 agonists that may be present in the contaminated batches (glycolipids and lipopeptides).

In one preferred embodiment of the methods according to the present invention, a first series of cell tests is carried out on nonfiltered samples, so as to measure the responses without taking into account the size of the molecules and to preserve the possible synergistic effects between these molecules. Then, in a second series, the cell tests are carried out on the fractions obtained by ultrafiltration (cut-off thresholds: 30 and 100 kDa), so as to verify whether the treatments modify the size of the contaminants (hydrolysis compared with aggregation), and/or do not remove certain contaminants of defined size.

In order to illustrate the method of the invention, various decontamination steps are carried out on various distinct glucose polymer matrices and a batch of glucose polymer hydrolysate:
glucose polymers, raw materials of icodextrin (before chromatographic fractionation according to the teaching of patent EP 667 356),
a batch of icodextrin,
a batch of branched maltodextrin, sold by the applicant company under the brand name NUTRIOSE® FB06,
a batch of dextrose monohydrate prepared so as to be conditioned in an injectable solution, sold by the applicant company under the brand name LYCADEX® PF,
a batch of highly-branched soluble glucose polymers, prepared according to the teaching of international patent application WO 2007/099212 of which the applicant company is the proprietor,
a commercial maltodextrin.

The treatments retained are:
heat treatment, for example:
   70° C. and/or
   120° C.,
acidification,
passing over activated carbon of various qualities, for example:
   SX+ from the company NORIT,
   SX2 from the company NORIT,
   C EXTRA USP from the company NORIT,
   A SUPRA EUR from the company NORIT,
   ENO-PC from the company CECA,
   L4S from the company CECA,
   L3S from the company CECA,
   CSA from the company CECA,
passing over adsorption resin,
   Amberlite XAD-4 from the company Rohm & Haas,
   Amberlite XAD-761 from the company Rohm & Haas,
   Amberlite XAD-1600 from the company Rohm & Haas,
   Amberlite XAD-16 HP(=FPX66) from the company Dow,
   Dowex SD2 from the company Dow,
   Macronet MN-100 from the company Purolite,
   Macronet MN-150 from the company Purolite,
ultrafiltration having a different cut-off threshold, for example
   5 kDa;
   30 kDa;
enzymatic hydrolysis using specific enzymes, for example:
   β-1,3-glucanase,
   proteases,
   enzymatic preparations with mannanase activity (for example Mannaway®, sold by the company NOVOZYMES, used for its detergent and clarifying properties),
   enzymatic preparations with endo-beta-glucanase activity (for example, SEBflo® Tl, produced by the company Specialty Enzymes and Biotechnologies Co. and sold by Advanced Enzyme Technologies Ltd.),
   enzymatic preparations with acid protease activity (for example, SEBPro® FL100, produced by the company Specialty Enzymes and Biotechnologies Co. and sold by Advanced Enzyme Technologies Ltd.).

The invention will be understood more clearly by means of the following examples, which are meant to be illustrative and nonlimiting.

### BRIEF DESCRIPTION OF THE FIGURES

FIG. 1: Raw-Blue™ cell responses to standard agonists.

FIG. 2: HEK-Blue™ TLR2 cell responses to standard agonists.

FIG. 3: HEK-Blue™ TLR4 cell responses to standard agonists.

FIG. 4: HEK-Blue™ NOD2 cell responses to standard agonists.

FIG. 5: HEK-Blue™ Null cell responses to standard agonists.

FIG. 6: Raw-Blue™ cell responses induced by nonfiltered matrices and after passing over 100 kDa and 30 kDa filters.

FIG. 7: HEK-Blue™ TLR2 cell responses induced by nonfiltered matrices and after passing over 100 kDa and 30 kDa filters.

FIG. 8: HEK-Blue™ TLR4 cell responses induced by nonfiltered matrices and after passing over 100 kDa and 30 kDa filters.

FIG. 9: HEK-Blue™ NOD2 cell responses induced by nonfiltered matrices and after passing over 100 kDa and 30 kDa filters.

FIG. 10: HEK-Blue™ Null cell responses induced by the matrices.

FIG. 11: Assessment of the inflammatory activities of the various matrices.

FIG. 12: Cell responses induced by the matrices after passing over carbon SX+.

FIG. 13: Cell responses induced by the E3063 matrix after passing over various carbons.

FIG. 14: Cell responses induced by the E1565 matrix after passing over various carbons.

FIG. 15: Cell responses induced by the E1242 matrix after passing over various carbons.

FIG. 16: Cell responses induced by the Lab3943 matrix after passing over various carbons.

FIG. 17: Cell responses induced by the E1565 matrix after treatment by ultrafiltration on 5 kDa.

FIG. 18: Cell responses induced by the Lab3943 matrix after treatment by ultrafiltration on 5 kDa.

FIG. 19: Cell responses induced by the E3063 and E5250 matrices after treatment with Mannaway®.

FIG. 20: Cell responses induced by the E5250 matrix after treatment with SEBflo® TL.

FIG. 21: Cell responses induced by the E3063 matrix after treatment with SEBPro® FL100.

FIG. 22: Cell responses induced by the E1565 matrix after treatment with industrial resins.

### EXAMPLES

### Example 1

### Establishment of the Dose-Response Curves

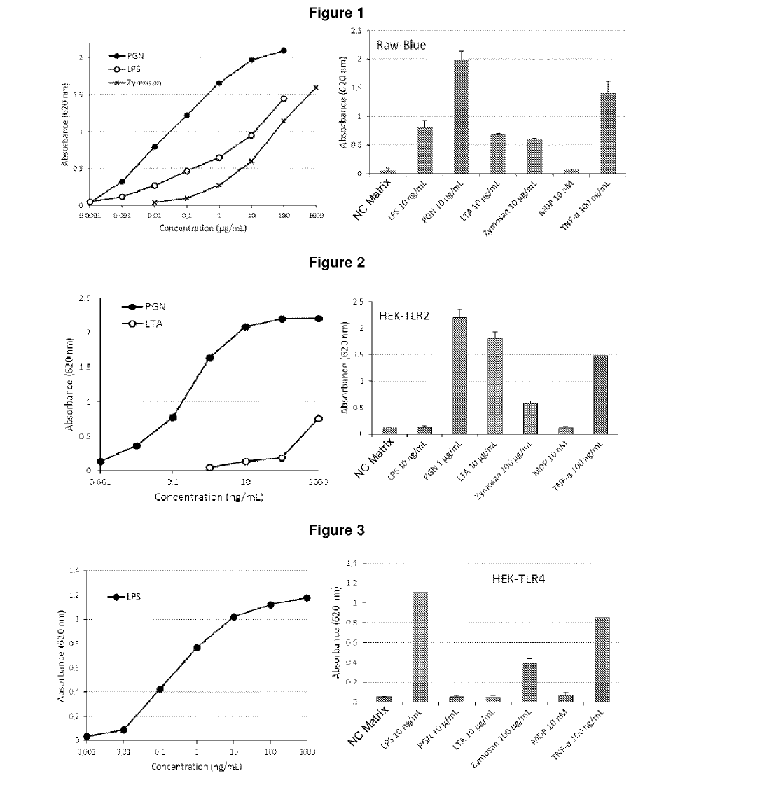

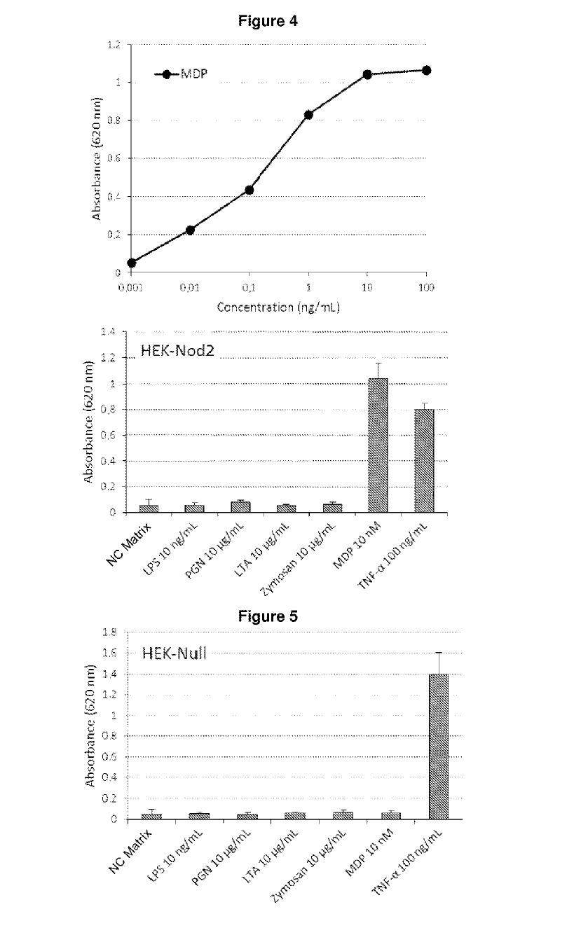

The dose-response curves are produced with standard agonist molecules: LPS, PGN, LTA, zymosan and MDP. The Raw-Blue™ and HEK-Blue™ TLR2, TLR4, NOD2 and Null lines are incubated with increasing concentrations of agonists, and the cell response is measured by quantifying the SEAP activity (FIGS. 1-5). TNF-α is used as a positive control for cell activation:
Raw-Blue™ line: the cells respond to the major inflammatory molecules that may be present in the glucose polymer matrices and derivatives (PGN, LPS, zymosan, LTA); they in particular have a strong reactivity with respect to PGNs, but do not respond to their depolymerization products (MDP).
HEK-Blue™ hTLR2 line: strong reactivity with respect to PGNs; the cells respond more weakly to the other TLR2 ligands (LTA, zymosan) and show no reactivity with respect to LPSs and to MDP.
HEK-Blue™ hTLR4 line: strong reactivity with respect to LPSs; the cells respond very weakly to zymosan and show no reactivity with respect to PGN, LTA and MDP.
HEK-Blue™ hNOD2 line: strong reactivity with respect to MDP.
HEK-Blue™ Null2 line: control for absence of cell toxicity.

### Example 2

### Preparation of the Various Distinct Glucose Polymer Matrices and of a Batch of Glucose Polymer Hydrolysate

As indicated above, the matrices are the following:
5 glucose polymers, raw materials of icodextrin (before chromatographic fractionation according to the teaching of patent EP 667 356), referenced here E1565, E3063, E1242, E5248 and E5250.

The preparation of these five polymers is carried out in accordance with the teachings of patent application WO 2012/059685;
a contaminated batch of icodextrin (referenced here E209J) and a “standard” batch of icodextrin, i.e. control for non-contamination in the cell tests (referenced here P11-11). These batches are prepared according to the teaching of patent EP 667 356, described in detail in Example 1 of patent application WO 2010/125315;
a batch of branched maltodextrin, sold by the applicant company under the brand name NUTRIOSE® FB06;
a batch of dextrose monohydrate, prepared so as to be conditioned in an injectable solution, sold by the applicant company under the brand name LYCADEX® PF;
a batch of highly-branched, soluble glucose polymers, for peritoneal dialysis, referenced here LAB3943.

This batch is prepared by double enzymatic treatment with branching enzyme and amyloglucosidase according to Example 2 of patent application WO 2007/099212.
A commercial maltodextrin (Cargill maltodextrin, C*Dry MD 01915, batch 02044770), referenced here Cargill.

### Example 3

### Analysis of the Cell Responses Induced by the Samples which are Nontreated or after Passing Over 100 kDa or 30 kDa Filter

The objective of these tests is to determine the pro-inflammatory reactivity and the nature of the contaminants present in the glucose polymer matrices and the batch of glucose polymer hydrolysate.

The samples according to Example 2 are prepared at 32% (weight/volume) in non-pyrogenic water (for injection).

The assays of the LPS and PGN levels were carried out prior to the cell tests using the SLP-HS and LAL assays (data presented below):

| | | | | | | Lab | Ico | | | |
|---|---|---|---|---|---|---|---|---|---|---|
| | P11-11 | E1242 | E1565 | E3063 | E5250 | 3943 | E209J | Cargill | NUTRIOSE ® | LYCADEX ® |
| PGN SLP | <3 | 21 | 2320 | 16185 | 4496 | 1263 | 393 | 2478 | 315 | <2 |
| (ng/g) -HS | | | | | | | | | | |
| LPS | <0.3 | 2.4 | 38.4 | 2.4 | 19.2 | 153.6 | 0.6 | 9.6 | >300 | <0.15 |
| (EU/g) LAL | | | | | | | | | | |
| LPS LAL | <0.3 | 1.2 | 4.8 | 1.2 | <0.3 | 153.6 | <0.3 | <0.3 | >300 | / |
| (EU/g) modifié | | | | | | | | | | |

For the cell tests, the samples are diluted to 1/10 in the cell culture medium (final concentration: 3.2% (w/v)).

The analyses are carried out on:
Raw-Blue™ line: any contaminants with high reactivity for PGNs,
HEK-Blue™ hTLR2 line: high reactivity for PGNs,
HEK-Blue™ hTLR4 line: high reactivity for LPSs,
HEK-Blue™ hNOD2 line: MDP and PGN depolymerization products,
HEK-Blue™ Null2 line: control for absence of cell toxicity.

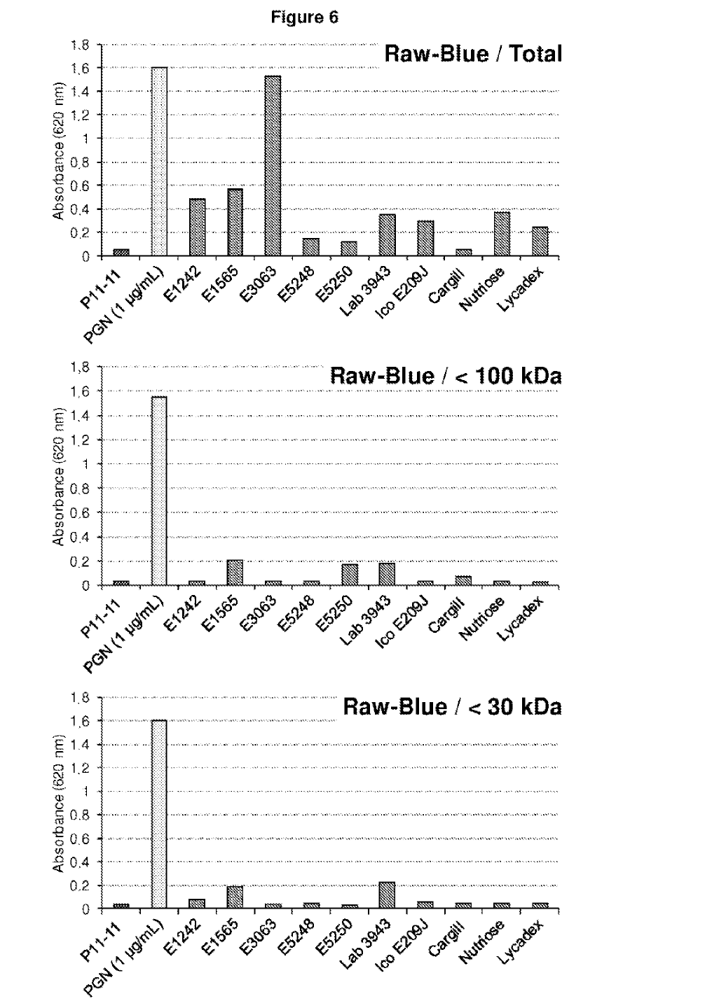

The results by cell type are presented in FIGS. 6 to 10.

Raw Cell Responses (FIG. 6):

With the exception of the Cargill matrix, which gives a response equivalent to that observed in the presence of the noncontamination control P11-11, all the other samples trigger an inflammatory response on contact with the macrophage line. The most reactive matrices are E-3063 (saturation of the cell response), then E-1242 and E-1565.

The contaminants are essentially molecules of high molecular weight (for example, PGN, zymosan) or capable of forming aggregates (for example, LPS, LTA). Indeed, the filtration at 100 kDa greatly reduced the responses induced by the samples, indicating that this treatment removed them to a large extent. Only the E-1242 and E-5250 matrices still have an activity significantly higher than that of P11-11, indicating that they contain contaminants having a size <100 kDa, probably originating from the degradation of larger contaminants. The filtration at 30 kDa is even more effective since the various samples lose virtually all their pro-inflammatory activity after treatment.

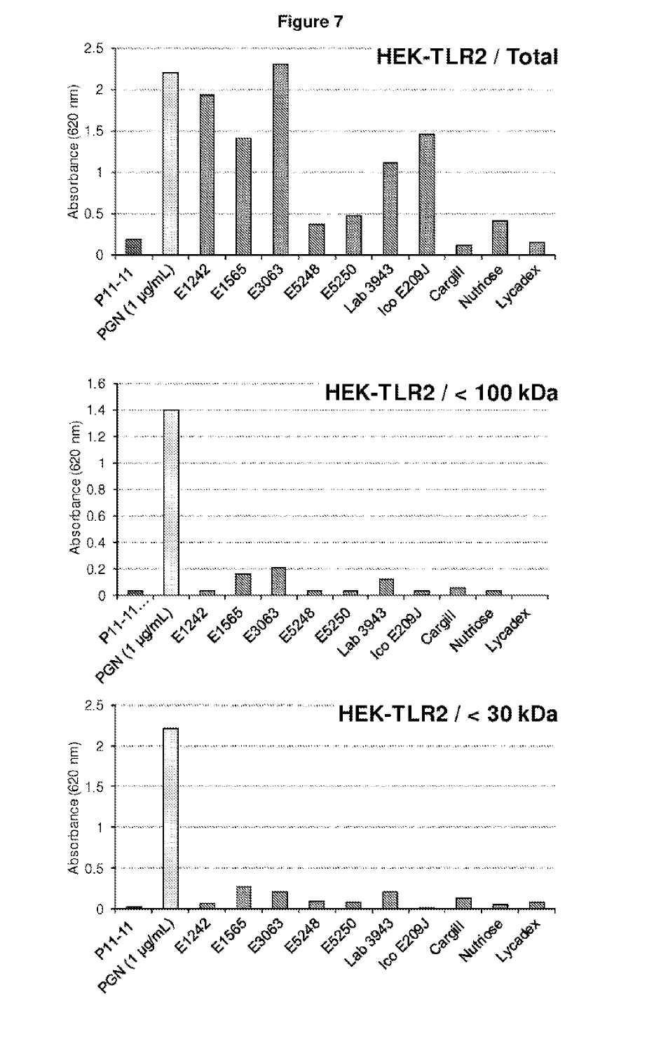

HEK-TLR2 Cell Responses (FIG. 7):

The results obtained with the HEK-TLR2 cells confirm the previous results.

The E-3063 matrix induces a saturated response, thereby indicating a very high TLR2 inducer contamination level. The E-1242, E-1565, Lab3943 and Ico-E209J matrices also give high responses, greater than those observed in the Raw cells. This difference is explained by the fact that they are loaded with strong inducers of TLR2 (PGNs or lipopeptides).

The filtrations at 100 kDa and at 30 kDa neutralize the inflammatory responses induced by these samples, indicating that the contaminants are predominantly high-molecular-weight PGNs. However, the E-3063 and E-1565 matrices still exhibit a significant activity after filtration. These data show that these compounds contain PGN degradation products and/or lipopeptides. Indeed, contrary to PGNs, the other strong inducers of TLR2 have a weight <30 kDa and would therefore still give a cell response after filtration.

The E-5250 and E-5248 matrices and the NUTRIOSE® give weak responses, of strength equivalent to that observed with the Raw cells, suggesting that these three samples contain weak inducers of TLR2 (for example, zymosan, J3-glucans or LTA).

As previously, the Cargill matrix and the LYCADEX® do not trigger responses, indicating the absence of TLR2-inducing contaminants.

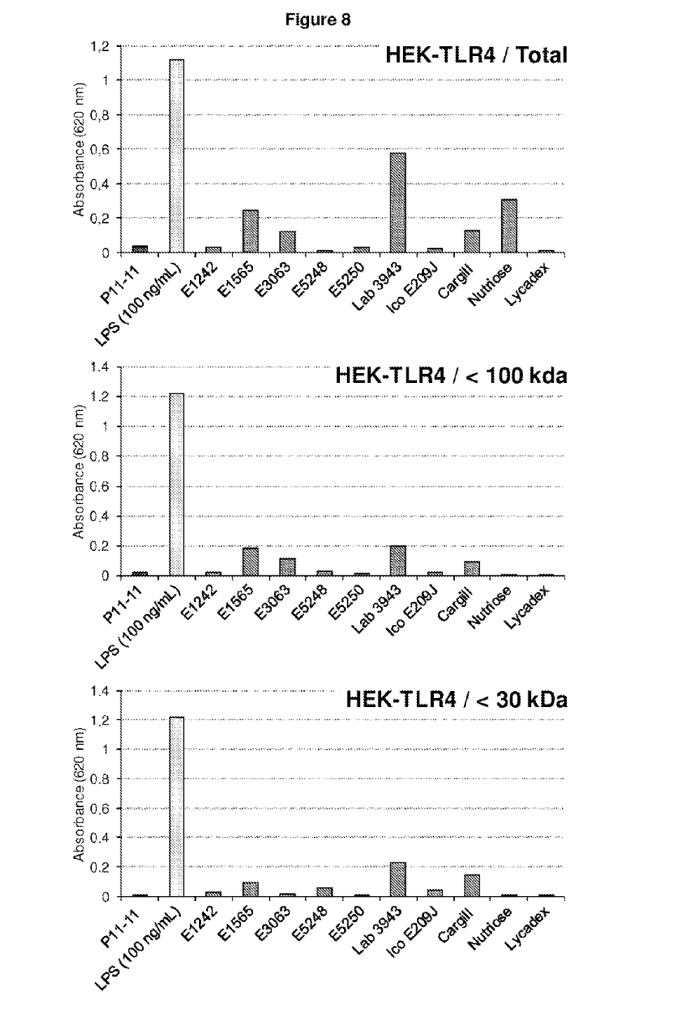

HEK-TLR4 Cell Responses (FIG. 8):

The E1565, E3063, Lab3943, Cargill and NUTRIOSE® matrices trigger a response of medium strength in the HEK-TLR4 cells, thereby confirming the presence of LPS. The filtrations partly reduce the responses of the cells, which can be explained by the fact that LPS can form aggregates, and that only the nonaggregated molecules were removed.

The E1242, IcoE209J, E5248, E5250 and LYCADEX® matrices do not trigger a significant response, indicating that the LPS levels are below the thresholds capable of triggering an inflammatory response. The first two matrices give an inflammatory response in the Raw cells, which correlates with a strong reactivity in the HEK-TLR2 cells. These data indicate that these two matrices are essentially contaminated with PGNs. The E5248 and E5250 matrices and the LYCADEX® also trigger an inflammatory response in the Raw cells. However, they are only barely or not at all active with respect to TLR2, thereby showing the presence of contaminants other than PGNs and LPSs in these three samples.

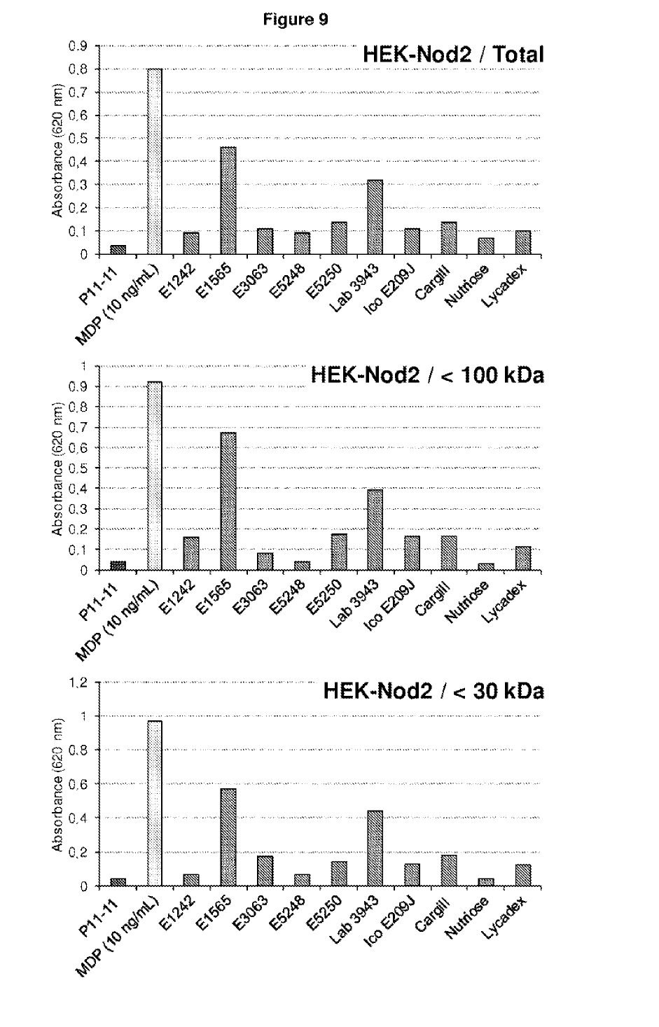

HEK-NOD2 Cell Responses (FIG. 9):

The HEK-NOD2 cells respond to all the samples, but only the E-1565, Lab3943 and Cargill matrices are strongly loaded with inducers of NOD2. This receptor reacts to the final product of PGN depolymerization (MDP), but also to the low-molecular-weight degradation products thereof. Consequently, the strength of the responses observed indicates that the samples are and/or were contaminated with PGN having undergone a more or less advanced degradation process. As expected, the filtrations at 100 and 30 kDa do not have a significant effect on the response of the cells, since the compounds in question (MDP and degraded PGNs) are of small size.

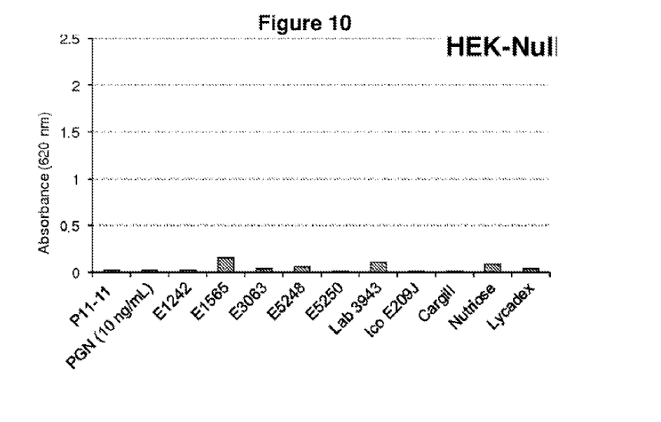

HEK-Null Cell Responses (FIG. 10):

Finally, the HEK-Null cells do not give significant responses in the presence of the various samples, proof that the reactivities observed in the other cell lines are not linked to a toxic effect, but indeed to a response of inflammatory type.

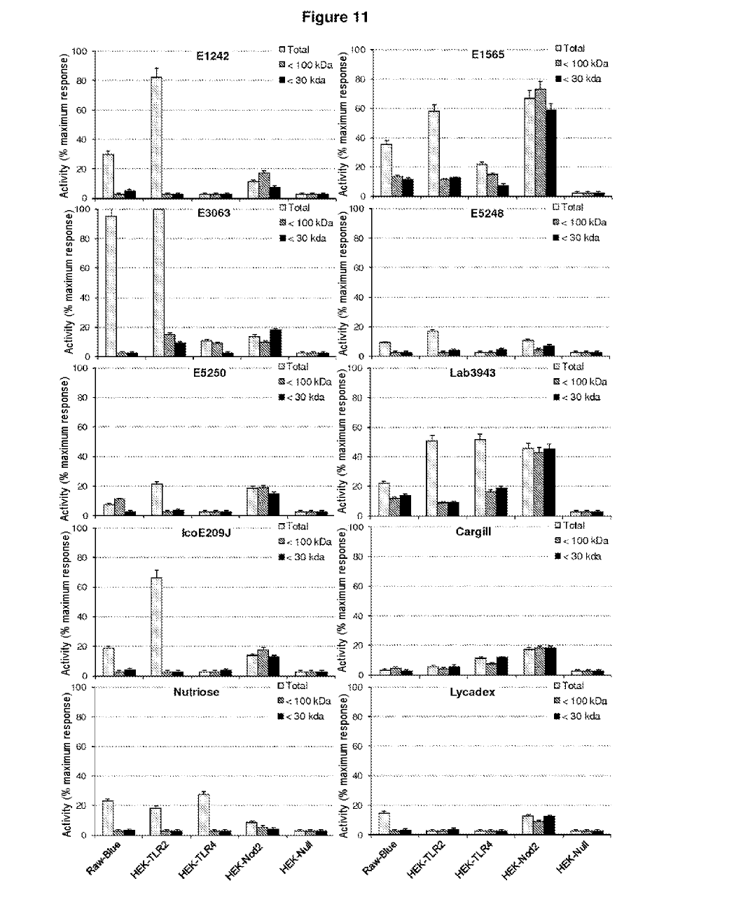

Assessment by Sample (FIG. 11):
E1242: medium-strength inflammatory activity linked to a strong contamination with slightly degraded PGNs.
E1565: medium-strength inflammatory activity linked to a strong contamination with partially degraded PGNs and to the presence of LPSs.
E3063: high-strength inflammatory activity linked to a very strong contamination with slightly degraded PGNs and to traces of LPSs.

E5248: low-strength inflammatory activity linked to a weak contamination with PGNs and to the presence of inflammatory molecules other than PGNs and LPSs.
E5250: low-strength inflammatory activity linked to a weak contamination with PGNs and to the presence of inflammatory molecules other than PGNs and LPSs.
Lab3943: medium-strength inflammatory activity linked to medium contaminations with partially degraded PGNs and with LPSs.
IcoE209J: medium-strength inflammatory activity linked to a strong contamination with slightly degraded PGN.
Cargill: absence of detectable inflammatory activity, but presence of PGN degradation products.
NUTRIOSE®: medium-strength inflammatory activity linked to traces of partially degraded PGNs and to a medium contamination with LPSs.
LYCADEX®: low-strength inflammatory activity linked to a weak contamination with PGN degradation products and to the presence of inflammatory molecules other than PGNs and LPSs.

### Example 4

### Effect of the Treatments by Passing Over Activated Carbons

In a first series of experiments, all the samples were subjected to two successive treatments with the same carbon (1% of NORIT SX+ carbon, at 80° C. for 1 h).

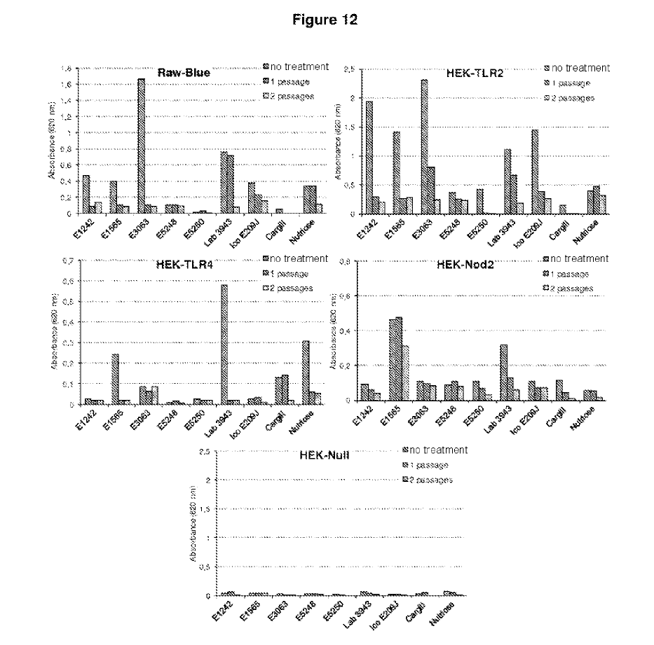

FIG. 12 presents the results obtained by cell line.

The first carbon treatment drastically decreases the capacity of the E1242, E-565, E3063 and IcoE209J samples to trigger an inflammatory response in the Raw and HEK-TLR2 cells. In any event, the second treatment further improves the removal of the molecules responsible for the inflammatory response. The effect of the treatment is much less marked for the E5248, E5250, and Lab3943 samples and the NUTRIOSE®. These data indicate that the treatment with the SX+ carbon would be effective for removing the barely degraded PGNs, thus reducing the inflammatory activity of the matrices for which these contaminants are predominant.

For the HEK-TLR4 cells, the response is very reduced for the E1565, Lab3943 and NUTRIOSE® matrices, which are the most contaminated with LPSs. However, the effect is not as marked as for the responses attributed to PGNs, showing kinetics specific to LPS.

The treatments with the SX+ carbon have little effect on the response of the HEK-NOD2 cells, thereby indicating that the PGN degradation products are not correctly removed. Finally, the HEK-Null cells are not reactive with respect to the samples treated, excluding any toxic effect associated with the carbon.

The differences observed after treatment by passing over activated carbon were expected, since the previous tests show that the samples contain contaminants of different molecular nature. An important piece of information provided by this experiment is the demonstration of a difference in effectiveness according to the size of the contaminants. Thus, the treatment with SX+ carbon would have a more marked effect on the removal of a certain category of contaminants, in particular high-molecular-weight PGNs.

In order to confirm this hypothesis, several carbons of different porosity were tested for their effectiveness in decontaminating the E3063, E1565, E1242 and Lab3943 matrices.

Contrary to the E3063 and E1242 matrices which are predominantly contaminated with PGNs of large size (strong TLR2 response), the E1565 and Lab3943 matrices contain partially degraded PGNs (TLR2 and NOD2 responses) and LPSs (TLR4 response).

The optimized treatment conditions are the following: carbon at 0.5%, pH adjusted to 4.5, incubation for 1 h at 80° C. After treatment, the samples are filtered through 0.22 μm filter and then used in the cell tests.

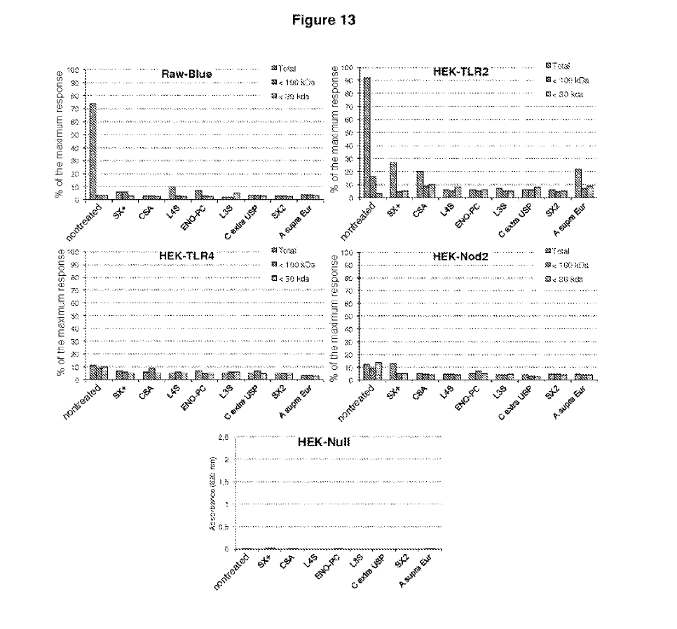

The results for the E3063 matrix are presented in FIG. 13.

The absence of response in the HEK-Null cells confirms that none of the carbons exhibit cell toxicity.

All the carbons tested are effective for drastically reducing the Raw and HEK-TLR2 cell responses, thereby attesting to their effectiveness for removing the large PGNs, which are the main contaminants of this matrix.

It is noted that the new carbons are equivalent to or more effective than the SX+.

Thus, the samples treated with L4S, L3S, ENO-PC, C-extra USP and SX2 induce cell responses close to the background noise in the HEK-TLR2 cells, thereby suggesting that these carbons are more effective for removing PGNs.

Regarding the less effective carbons, the filtration on 30 kDa and 100 kDa reduces the residual responses after treatment, which is proof that they were due essentially to traces of unremoved PGNs.

The same results are observed in the Raw cells, with the exception of L4S, which appears to be less effective, and A-Supra-Eur, which, on the contrary, is found to be more effective than in the HEK-TLR2 cells. The latter carbon could therefore have a broader spectrum of action and remove the other molecules, such as LPSs.

Even though the carbons significantly reduce the reactivity of the HEK-NOD2 and HEK-TLR4 cells with respect to the E3063 matrix, it is difficult to distinguish differences in effectiveness between the treatments because of the small size of the responses induced by this matrix in the two cell types.

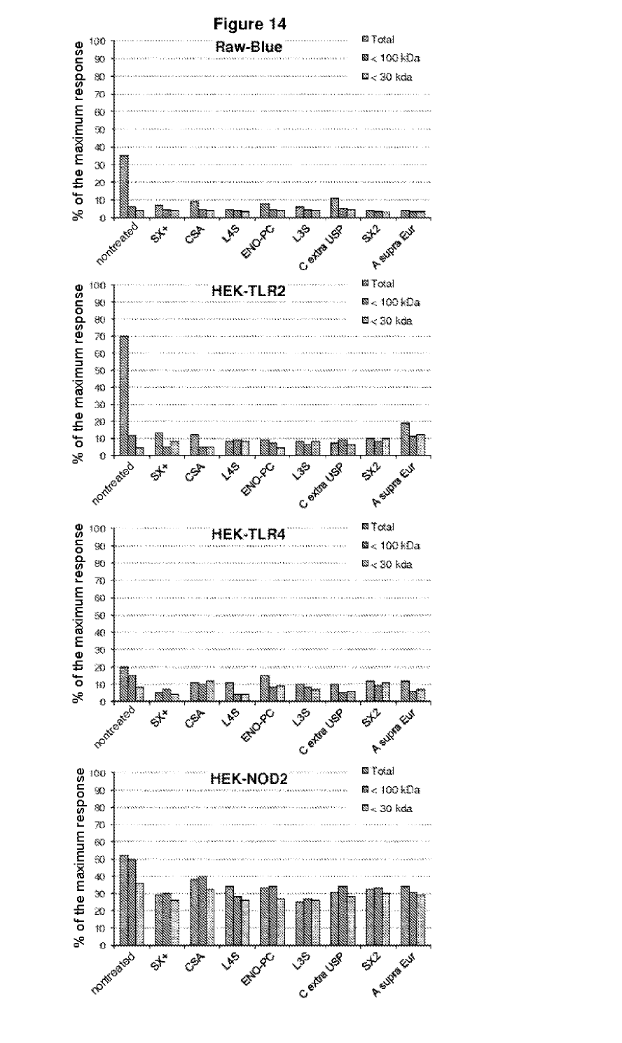

The results for the E1565 matrix are presented in FIG. 14.

As for the E3063 matrix, the same carbons are effective for reducing the HEK-TLR2 and Raw cell responses induced by E1565. However, a few minor differences can be noted, probably due to differences in sizes and therefore in properties of the PGNs.

Virtually the entire HEK-NOD2 cell response is clearly due to the presence of PGN degradation products, since the filtration at 30 kDa retains the contaminants only very slightly or not at all. On the other hand, the carbons are not very effective for reducing the response of these cells. Indeed, the decrease in the response caused by the reduction in the load of contaminants of MDP type does not exceed 50% of the maximum response of the cells.

Finally, the carbons have a medium effect on the TLR4 response. With the exception of SX+ which is very effective for removing all the forms of LPS, the reduction caused by the treatment with other carbons does not exceed 50% of the response induced by the same sample which has not been treated. However, it can be noted that the L4S and A-Supra-Eur carbons, and to a lesser extent the C-extra USP and ENO-PC carbons, are more effective for reducing the responses induced by molecules of size <100 kDa, thereby suggesting that these carbons preferentially act on nonaggregated LPSs.

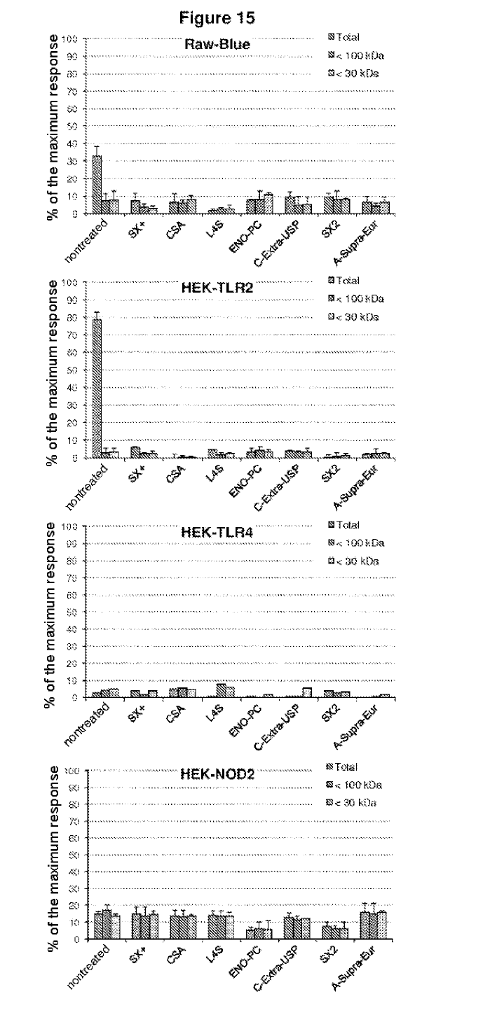

The results for the E1242 matrix are presented in FIG. 15.

All the carbons tested are effective for reducing the responses induced by the E1242 matrix in the Raw and HEK-TLR2 cells. The responses obtained, which are close or equal to the background noise, are identical before and after filtration on 30 kDa and 100 kDa, which is proof that the large molecules corresponding to PGN have been removed.

The E1242 matrix is very weakly contaminated with LPS. However, it can be noted that ENO-PC and A-Supra-Eur are more effective than the other carbons for decreasing the HEK-TLR4 cell response to the level of the background noise. This observation confirms that these two carbons have a broad spectrum of action and are effective for removing molecules other than PGNs, such as LPSs.

Finally, the carbons are not effective for removing PGN degradation products, with the exception of ENO-PC and SX2, which reduce the HEK-NOD2 cell response by approximately 50%.

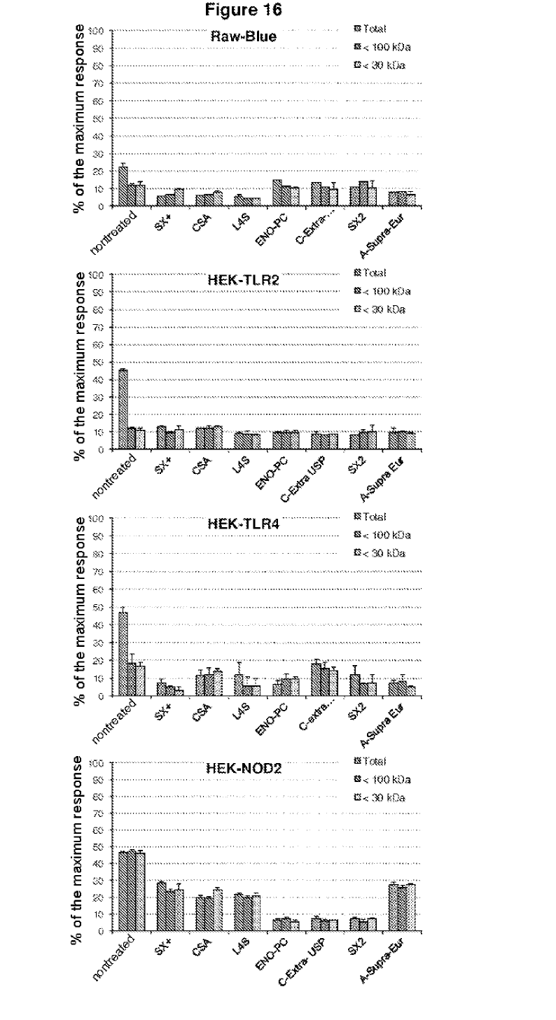

The results for the Lab3943 matrix are presented in FIG. 16.

This matrix is contaminated with a broad spectrum of different molecules. As expected, all the carbons reduce the responses induced in the Raw cells, but with different effectivenesses. It can be noted that SX+, CSA, L4S and, to a lesser extent, A-Supra-Eur are the most effective, thereby confirming that these carbons have a broad spectrum of action. For the HEK-TLR2 cells, the carbons are all found to be effective for removing PGNs, but the residual responses remain identical before and after filtration, indicating that the PGN degradation products are more difficult to remove.

The behavior of the carbons in the removal of LPSs is also variable. Thus, the SX+, ENO-PC, L4S and A-Supra-Eur carbons are still the most effective for reducing the HEK-TLR4 cell response. Finally, only the ENO-PC, C-Extra-USP and SX2 carbons are found to be relatively active for strongly reducing the HEK-NOD2 cell response, which is proof that they are effective for removing the PGN degradation products present in this matrix.
C-extra-USP and SX2: effective for removing PGNs and the degradation products thereof.
A-Supra-Eur: broad spectrum with higher effectiveness for high-molecular-weight molecules (for example: aggregated LPSs and PGNs).
ENO-PC: broad spectrum with higher effectiveness for molecules having a molecular weight <100 kDa (for example: LPSs and PGN degradation products).
other carbons: spectra of action and effectiveness at most equivalent to those of the SX+ carbon.

### Example 5

### Effect of a Treatment by Ultrafiltration on 5 kDa

The objective of the treatment by ultrafiltration is to reduce, or even remove, the contamination with molecules of small size, so as to counter their participation in the triggering of an inflammatory response, whether it is via a direct effect or via a phenomenon of synergy with other contaminating molecules.

The experiments were carried out on the E1565 and Lab3943 matrices, which are both contaminated with partially degraded PGNs (TLR2 and NOD2 responses) and LPSs (TLR4 response).

The filtration on 5 kDa was carried out at an average flow rate of 25 ml/min. The filtrate flow rates are respectively 55 ml/h for E1565 and 65 ml/h for Lab3943.

In order to verify the effectiveness of the ultrafiltration, the cell responses were first measured using samples originating from the retentate and filtrate fractions recovered after passage of the starting solution (100 ml).

The ultrafiltration was then carried out in closed circuit with continuous injection of the retentate into the starting sample. In order to compensate for the loss of liquid due to the removal of the filtrate, the volume of the sample was continuously adjusted to the starting volume by adding water for injection. In this case, the cell tests were carried out using specimens taken from the sample after 1 h, 2 h and 3 h of ultrafiltration.

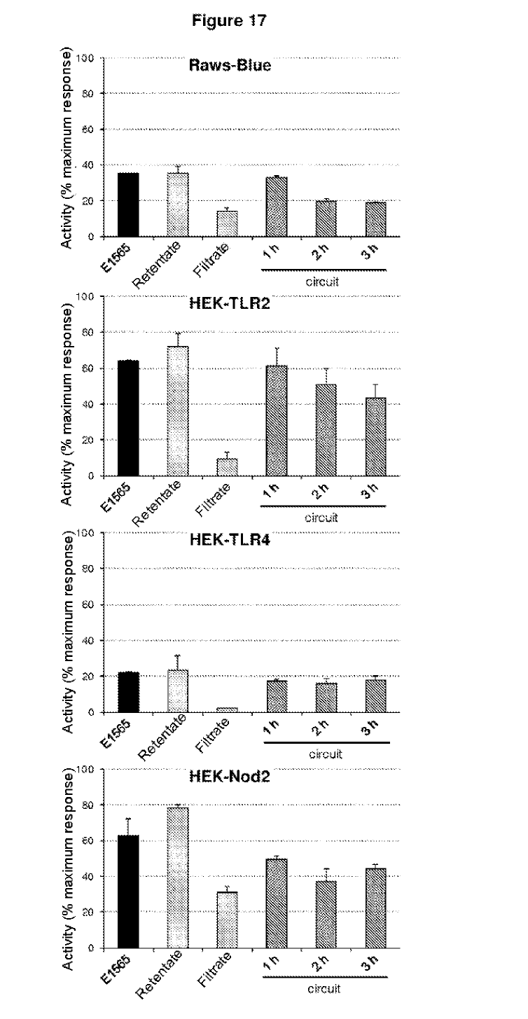

The results for the E1565 matrix are presented in FIG. 17.

The responses induced by the retentate fractions remain similar to those observed for the nonfiltered samples in the four cell tests. However, a significant inflammatory response is observed in response to the filtrate fractions in the Raw and HEK-NOD2 cells. These data are compatible with the cut-off threshold of the filter (5 kDa), which allows the PGN depolymerization products to pass, but not the PGNs and the LPS, especially if the latter are in the form of aggregates. On the other hand, the absence of a decrease in inflammatory response in the retentates indicates that a single passage through the filter is ineffective for reducing the inflammatory reactivity of the matrix. This is because the division between the retentate/filtrate fractions is 25 to 1, which is insufficient to remove the small inflammatory molecules.

The continuous ultrafiltration is effective for decreasing the response induced in the HEK-NOD2 cells, which was predictable, but also in the Raw and HEK-TLR2 cells. Given the contamination of E1565 with PGNs and LPS, the decrease in response in the latter two cell types is certainly linked to a reduction in the synergistic activity of the small inflammatory molecules.

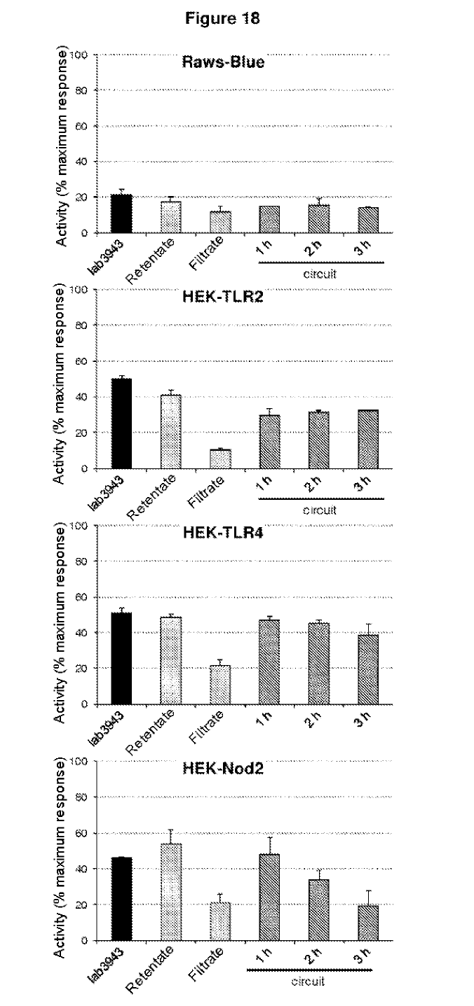

The results for the Lab3943 matrix are presented in FIG. 18.

As expected, an inflammatory activity associated with MDP is found in the filtrate (HEK-NOD2). A significant response is also observed in the HEK-TLR4 cells. This result shows that the LPS is in a structural configuration that is less aggregated than that present in the E1565 matrix, thereby allowing it to pass through the filter.

As for E1565, the continuous filtration is effective for decreasing the PGN depolymerization product load, which is visible through a clear reduction in the HEK-NOD2 cell response (direct effect) and through a smaller but significant decrease in the reactivity of the Raw and HEK-TLR2 cells (synergistic action).

### Example 6

### Effect of the Enzymatic Treatments

The objective of these tests is to test the capacity of industrial enzymes to decrease the pro-inflammatory reactivity of the contaminants present in glucose polymer matrices.

The samples are prepared at 32% (weight/volume) and treated in the presence of the enzymes according to the conditions described hereinafter. After treatment, the enzymes are deactivated by heating, and the solutions are filtered through a sterile 0.22 μm filter and then used in the cell tests.

Three industrial enzymatic preparations were tested for their capacity to reduce the contaminant load:
Mannaway®: incubation at 0.4% (vol/vol), pH 10, 50° C., for 4 h and 24 h.
SEBflo® TL: incubation at 0.35 mg/g of matrix, 50° C., pH 5, from 30 min to 24 h.
SEBPro® FL100: incubation at 4% (vol/vol), pH 3, 55° C., from 1 h to 24 h.

The two matrices chosen for the tests are: E3063 (strong contamination with slightly degraded PGNs and traces of LPS) and E5250 (weak PGN contamination and presence of inflammatory molecules other than PGNs and LPSs).

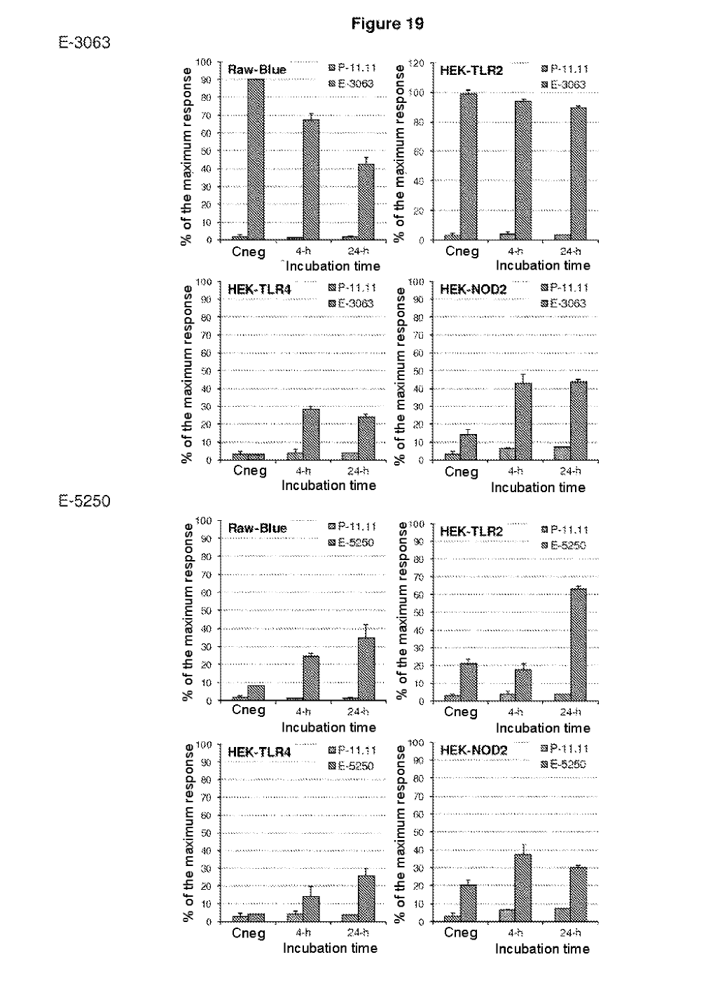

The effects of the treatments of the two matrices with Mannaway® are presented in FIG. 19.

The addition of the enzymatic solution to the P-11.11 matrix (noncontamination control) induces no inflammatory response in the Raw, HEK-TLR2 or HEK-TLR4 cells. A slight increase is observed in the HEK-NOD2 cells, suggesting the presence of PGN degradation products in trace amounts. However, these results show that the enzyme used under the conditions in the experiment does not introduce major pro-inflammatory contaminants.

The tests carried out on the E3063 matrix show that the enzyme probably has a weak lytic action on PGNs, since a decrease in the responses in the Raw and HEK-TLR2 cells is observed. The treatment causes an increase in the HEK-NOD2 cell response, which is compatible with a partial degradation of PGNs. On the other hand, an increase in the HEK-TLR4 cell response is also observed. Given that the enzymatic preparation does not introduce any contamination, the appearance of LPS is probably due to a release of this contaminant from the matrix itself.

The enzymatic treatment of the E5250 matrix induces an increase in the inflammatory response in the four cell types. Originally, this matrix triggered weak inflammatory responses. Consequently, the increase in the TLR2 and TLR4 responses suggests that the enzyme released contaminants of PGN and LPS type from the matrix.

Mannaway® is commonly used as an agent for clarifying food-processing products. The results obtained suggest that the enzyme probably dissociated macrocomplexes (bacterial debris) which were normally removed by the step of filtration through a 0.22 μm filter. By solubilizing these inflammatory molecules, the enzyme made them accessible for inducing responses in the cell tests.

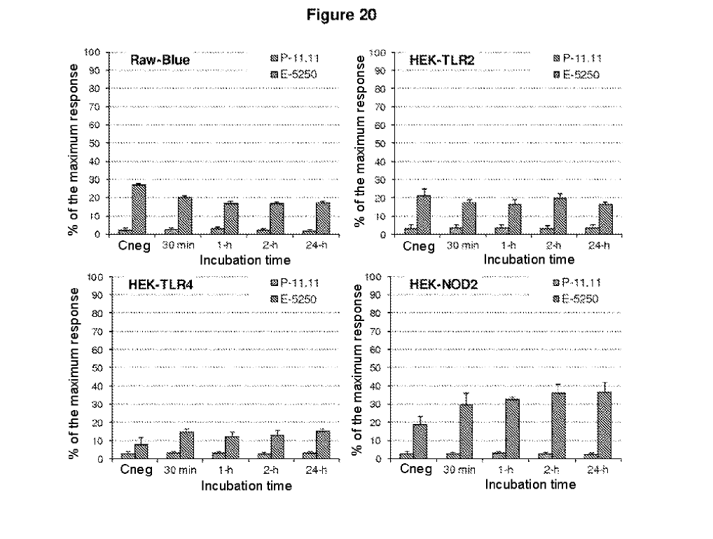

The effects of the treatments of the E5250 matrix with SEBflo® TL are presented in FIG. 20.

The addition of the enzymatic solution to the P-11.11 matrix induces no inflammatory response in the four cell types, which is proof that the enzyme used under the conditions of the experiment does not introduce contaminants.

The treatment of the E5250 matrix induces a slight decrease in the inflammatory responses in the Raw and HEK-TLR2 cells. The enzyme is described essentially for its beta-glucanase properties. However, the decrease in the cell responses observed is accompanied by an increase in the HEK-NOD2 cell response. These data indicate that the enzymatic preparation also contains an activity capable of degrading PGNs.

In parallel to the appearance of the PGN degradation products, a slight increase in the TLR4 response is observed in the presence of the enzyme, even though the latter is not contaminated. As previously, the enzyme probably released inflammatory contaminants, but to a lesser degree than what is observed with Mannaway®.

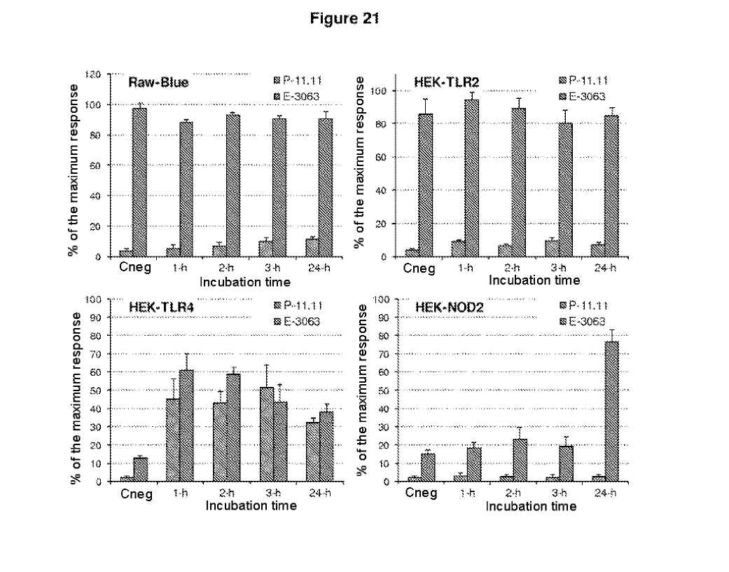

The effects of the treatments of the E3063 matrix with SEBPro® FL100 are presented in FIG. 21.

The addition of the enzymatic preparation to the P-11.11 matrix induces a strong inflammatory response in the HEK-TLR4 cells, and also weak but significant responses in the Raw and HEK-TLR2 cells. These data indicate that the enzyme is contaminated with LPS and traces of PGN.

The addition of the enzyme to the E3063 matrix induces a slight decrease in the TLR2 response, which is accompanied by an increase in the HEK-NOD2 cell response. These data indicate that SEBPro® FL100 has a weak lytic action on PGNs. However, its use would necessarily require a prior decontamination step in order to remove the LPS.

### Example 7

### Effect of the Treatments by Passing Over Resins

The objective of these tests is to test the capacity of industrial resins to retain the contaminants present in glucose polymer matrices and, consequently, to reduce the pro-inflammatory reactivity of these matrices.

The tests were carried out with the E1565 matrix (solubilized at 32% weight/volume in sterile water), since it is contaminated with the various types of pro-inflammatory molecules that may be found in production circuits (TLR2, TLR4 and NOD2 responses).

For the experiments, the solution to be decontaminated was continuously eluted on a column containing 20 ml of each resin (bed volume). The cell tests for inflammatory reactivity were carried out using the solution before it was passed over the column (contamination control), and then on the samples recovered after passing 4 volumes (passage 5) and 10 volumes (passage 11) of solution. This procedure made it possible to verify whether the presence of the glucose polymer did not cause a resin saturation phenomenon.

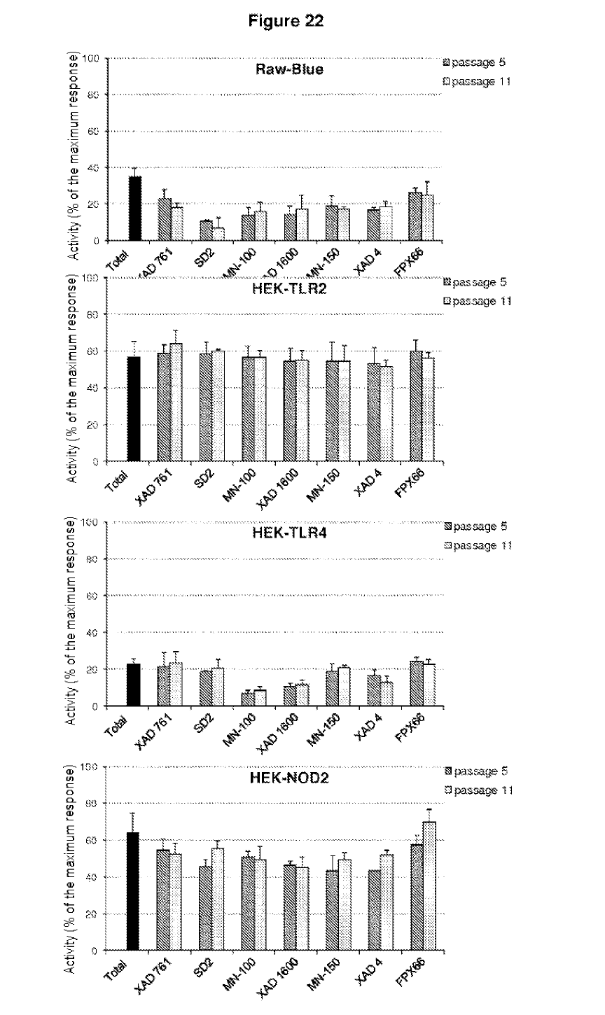

The results are presented in FIG. 22.

The passing of the solution over the various resins causes a decrease in the inflammatory reactivity of the E1565 matrix on contact with the Raw cells. With the exception of FPX66, the decrease in response reaches at least 50% for the other resins. The reduction even reaches 70% after passing over the SD2 resin, thereby indicating that this resin is the most effective for removing the contaminating molecules with pro-inflammatory activity contained in the E1565 matrix. In any event, no significant difference is observed between passages 5 and 11, thereby excluding any substrate saturation phenomenon.

The reactivity of the HEK-TLR2 cells with respect to the E5250 matrix is not significantly modified after passing over the various resins, thereby indicating that these treatments are ineffective for reducing the contaminated PGN load.

The MN-100 and XAD-1600 resins are, for their part, found to be very effective for reducing the HEK-TLR4 responses with respect to the E1565 matrix. These data indicate that these two resins have a high capacity for retaining molecules of LPS type. Conversely, the other resins are barely, or even totally, ineffective for retaining this contaminant.

Finally, the various resins moderately reduce the HEK-NOD2 cell responses with respect to the matrix, with the exception of FPX66, which is totally ineffective. However, the effect observed remains weak, demonstrating that the resins have a weak capacity for retaining PGN degradation products.

Besides the presence of traces of LPS, the E1565 matrix is strongly contaminated with PGN and with degradation products thereof. The latter have little inflammatory reactivity per se; on the other hand, they are capable of acting in synergy with the other inflammatory molecules that interact with TLRs, such as PGNs and LPSs, and of exacerbating the overall immune response.

The tests carried out in this example show a significant decrease in the reactivity of the Raw cells after passing over the various resins. This decrease in overall inflammatory response is not subsequent to a retention of PGNs, since the passing over resins does not modify the TLR2 responses.

Only two resins (MN-100, XAD-1600) out of seven are clearly effective for reducing the TLR4 response. It can therefore be deduced therefrom that the decrease in inflammatory response observed in the Raw cells is at least partially subsequent to the retention of LPS after passing the matrix over these two resins.

With the exception of FPX66, all the resins moderately retain the PGN degradation products. Given the impact of these small molecules on the exacerbation of inflammatory responses, these results indicate that the major effect of the resins is to remove the synergistic effects associated with these small molecules.

As for the FPX66 matrix, its effect is probably linked to the removal of inflammatory molecules other than LPS, and PGNs and degradation products thereof. This hypothesis is compatible with the fact that this resin is the least effective for reducing the overall inflammatory response.

As a whole, these data indicate that treatment of the matrices with certain judiciously selected industrial resins can prove to be effective for removing inflammatory contaminants other than PGNs, but also reducing the effects of synergy observed between these molecules. Example 4 of the present study showed that some industrial carbons are particularly effective for removing PGNs. Consequently, a procedure combining the two types of treatment should make it possible to target the various families of contaminants and to provide matrices free of inflammatory reactivity.

1-13. (canceled)

14. A method for testing the effect of a production step or production steps or the effectiveness of a purification step or purification steps on the presence or the nature of pro-inflammatory molecules in glucose polymers or hydrolysates thereof, comprising:
a) providing glucose polymers or hydrolysates thereof;
b) optionally, detecting or assaying the pro-inflammatory molecules in the glucose polymers or hydrolysates thereof provided in step a);
c) carrying out the production or purification step or steps on the glucose polymers or hydrolysates thereof provided in step a);
d) detecting or assaying the pro-inflammatory molecules in the glucose polymers or hydrolysates thereof obtained after step c);
e) determining the effectiveness or the impact of step c) on the presence or the nature of the pro-inflammatory molecules;
in which the step for detecting or assaying the pro-inflammatory molecules in the glucose polymers or hydrolysates thereof comprises an in vitro inflammatory response test using a cell line, the cell line being either a macrophage or a macrophage-differentiated cell line, or a cell expressing one or more TLR (Toll Like Receptor) or NOD (Nucleotide-binding Oligomerization Domain-containing protein) receptors and making it possible to detect the responses of the receptor or receptors, or a combination thereof.

15. The method of claim 14, wherein the in vitro inflammatory response test comprises bringing the glucose polymers or hydrolysates thereof into contact with the MDP- or LPS-sensitized, macrophage-differentiated THP-1 cell line, the pro-inflammatory molecules being detected or assayed by measuring the amount of RANTES or TNF-α produced by the cell line.

16. The method of claim 14, wherein the in vitro inflammatory response test comprises bringing the glucose polymers or hydrolysates thereof into contact with a macrophage line transfected with a reporter gene, the transcription of which is under the direct control of the inflammatory signaling pathways, the pro-inflammatory molecules being detected or assayed by measuring the activity or the signal of the reporter gene.

17. The method of claim 14, wherein the in vitro inflammatory response test comprises bringing the glucose polymers or hydrolysates thereof into contact with a cell line expressing the TLR2 receptor and a reporter gene, the transcription of which is under the direct control of the TLR2 signaling pathways, the pro-inflammatory molecules being detected or assayed by measuring the activity or the signal of the reporter gene.

18. The method of claim 14, wherein the in vitro inflammatory response test comprises bringing the glucose polymers or hydrolysates thereof into contact with a cell line expressing the TLR4 receptor and a reporter gene, the transcription of which is under the direct control of the TLR4 signaling pathways, the pro-inflammatory molecules being detected or assayed by measuring the activity or the signal of the reporter gene.

19. The method of claim 14, wherein the in vitro inflammatory response test comprises bringing the glucose polymers or hydrolysates thereof into contact with a cell line expressing the NOD2 receptor and a reporter gene, the transcription of which is under the direct control of the NOD2 signaling pathways, the pro-inflammatory molecules being detected or assayed by measuring the activity or the signal of the reporter gene.

20. The method of claim 14, wherein the in vitro inflammatory response test comprises bringing the glucose polymers or hydrolysates thereof into contact with:
a) the MDP- or LPS-sensitized, macrophage-differentiated THP-1 cell line, the pro-inflammatory molecules being detected or assayed by measuring the amount of RANTES or TNF-α produced by the cell line; and/or
b) a macrophage line transfected with a reporter gene, the transcription of which is under the direct control of the inflammatory signaling pathways, the pro-inflammatory molecules being detected or assayed by measuring the activity or the signal of the reporter gene; and/or
c) a cell line expressing the TLR2 receptor and a reporter gene, the transcription of which is under the direct control of the TLR2 signaling pathways, the pro-inflammatory molecules being detected or assayed by measuring the activity or the signal of the reporter gene; and/or
d) a cell line expressing the NOD2 receptor and a reporter gene, the transcription of which is under the direct control of the NOD2 signaling pathways, the pro-inflammatory molecules being detected or assayed by measuring the activity or the signal of the reporter gene; and/or
e) a cell line expressing the TLR4 receptor and a reporter gene, the transcription of which is under the direct control of the TLR4 signaling pathways, the pro-inflammatory molecules being detected or assayed by measuring the activity or the signal of the reporter gene; and/or
f) a control line not transfected with an immunity receptor.

21. The method of claim 14, wherein the in vitro inflammatory response test comprises bringing the glucose polymers or hydrolysates thereof into contact with:
a) a macrophage line transfected with a reporter gene, the transcription of which is under the direct control of the inflammatory signaling pathways, the pro-inflammatory molecules being detected or assayed by measuring the activity or the signal of the reporter gene;
b) a cell line expressing the TLR2 receptor and a reporter gene, the transcription of which is under the direct control of the TLR2 signaling pathways, the pro-inflammatory molecules being detected or assayed by measuring the activity or the signal of the reporter gene; and/or
c) a cell line expressing the TLR4 receptor and a reporter gene, the transcription of which is under the direct control of the TLR4 signaling pathways, the pro-inflammatory molecules being detected or assayed by measuring the activity or the signal of the reporter gene; and/or
d) a cell line expressing the NOD2 receptor and a reporter gene, the transcription of which is under the direct control of the NOD2 signaling pathways, the pro-inflammatory molecules being detected or assayed by measuring the activity or the signal of the reporter gene; and
e) a control line not transfected with an immunity receptor.

22. The method of claim 14, wherein the pro-inflammatory molecules are molecules of bacterial origin.

23. The method of claim 14, wherein the production or purification step or steps is or are chosen from steps of heat treatment, of acidification, of passing over activated carbon, of passing over adsorption resins, of ultrafiltration, of filtration, or of chemical or enzymatic hydrolysis, or combinations thereof.

24. The method of claim 14, wherein the glucose polymers are selected from icodextrin and branched or unbranched maltodextrins, and the glucose polymer hydrolysates are a product of total hydrolysis.

25. The method of claim 14, wherein the samples of glucose polymers or of hydrolysates thereof are prefiltered with a cut-off threshold at 30 kDa, and the filtrate is brought into contact with the cell line used in the test.

26. The method of claim 22, wherein said pro-inflammatory molecules of bacterial origin are PGNs, LPSs, lipopeptides, PGN depolymerization products MDP, formylated microbial peptides or f-MLP or β-glucans.

27. An optimized method for producing or purifying glucose polymers or hydrolysates thereof, comprising:
a) providing glucose polymers or hydrolysates thereof;
b) detecting or assaying the pro-inflammatory molecules in the glucose polymers or hydrolysates thereof provided in step a);
c) selecting the step or steps for producing or purifying the glucose polymers or hydrolysates thereof that is or are suitable for the pro-inflammatory molecules present in the glucose polymers or hydrolysates thereof;
d) optionally, carrying out the selected production or purification step or steps on the glucose polymers or hydrolysates thereof provided in step a); and
e) optionally, detecting or assaying the pro-inflammatory molecules in the glucose polymers or hydrolysates thereof obtained after step d);
in which the step for detecting or assaying the pro-inflammatory molecules in the glucose polymers or hydrolysates thereof comprises an in vitro inflammatory response test using a cell line, the cell line being either a macrophage or a macrophage-differentiated cell line, or a cell expressing one or more TLR (Toll Like Receptor) or NOD (Nucleotide-binding Oligomerization Domain-containing protein) receptors and making it possible to detect the responses of the receptor or receptors, or a combination thereof.

28. The method of claim 27, wherein the in vitro inflammatory response test comprises bringing the glucose polymers or hydrolysates thereof into contact with the MDP- or LPS-sensitized, macrophage-differentiated THP-1 cell line, the pro-inflammatory molecules being detected or assayed by measuring the amount of RANTES or TNF-α produced by the cell line.

29. The method of claim 27, wherein the in vitro inflammatory response test comprises bringing the glucose polymers or hydrolysates thereof into contact with a macrophage line transfected with a reporter gene, the transcription of which is under the direct control of the inflammatory signaling pathways, the pro-inflammatory molecules being detected or assayed by measuring the activity or the signal of the reporter gene.

30. The method of claim 27, wherein the in vitro inflammatory response test comprises bringing the glucose polymers or hydrolysates thereof into contact with a cell line expressing the TLR2 receptor and a reporter gene, the transcription of which is under the direct control of the TLR2 signaling pathways, the pro-inflammatory molecules being detected or assayed by measuring the activity or the signal of the reporter gene.

31. The method of claim 27, wherein the in vitro inflammatory response test comprises bringing the glucose polymers or hydrolysates thereof into contact with a cell line expressing the TLR4 receptor and a reporter gene, the transcription of which is under the direct control of the TLR4 signaling pathways, the pro-inflammatory molecules being detected or assayed by measuring the activity or the signal of the reporter gene.

32. The method of claim 27, wherein the in vitro inflammatory response test comprises bringing the glucose polymers or hydrolysates thereof into contact with a cell line expressing the NOD2 receptor and a reporter gene, the transcription of which is under the direct control of the NOD2 signaling pathways, the pro-inflammatory molecules being detected or assayed by measuring the activity or the signal of the reporter gene.

33. The method of claim 27, wherein the in vitro inflammatory response test comprises bringing the glucose polymers or hydrolysates thereof into contact with:
a) the MDP- or LPS-sensitized, macrophage-differentiated THP-1 cell line, the pro-inflammatory molecules being detected or assayed by measuring the amount of RANTES or TNF-α produced by the cell line; and/or
b). a macrophage line transfected with a reporter gene, the transcription of which is under the direct control of the inflammatory signaling pathways, the pro-inflammatory molecules being detected or assayed by measuring the activity or the signal of the reporter gene; and/or
c) a cell line expressing the TLR2 receptor and a reporter gene, the transcription of which is under the direct control of the TLR2 signaling pathways, the pro-inflammatory molecules being detected or assayed by measuring the activity or the signal of the reporter gene; and/or
d) a cell line expressing the NOD2 receptor and a reporter gene, the transcription of which is under the direct control of the NOD2 signaling pathways, the pro-inflammatory molecules being detected or assayed by measuring the activity or the signal of the reporter gene; and/or
e) a cell line expressing the TLR4 receptor and a reporter gene, the transcription of which is under the direct control of the TLR4 signaling pathways, the pro-inflammatory molecules being detected or assayed by measuring the activity or the signal of the reporter gene; and/or
f) a control line not transfected with an immunity receptor.

34. The method of claim 27, wherein the in vitro inflammatory response test comprises bringing the glucose polymers or hydrolysates thereof into contact with:
a) a macrophage line transfected with a reporter gene, the transcription of which is under the direct control of the inflammatory signaling pathways, the pro-inflammatory molecules being detected or assayed by measuring the activity or the signal of the reporter gene;
b) a cell line expressing the TLR2 receptor and a reporter gene, the transcription of which is under the direct control of the TLR2 signaling pathways, the pro-inflammatory molecules being detected or assayed by measuring the activity or the signal of the reporter gene; and/or
c) a cell line expressing the TLR4 receptor and a reporter gene, the transcription of which is under the direct control of the TLR4 signaling pathways, the pro-inflammatory molecules being detected or assayed by measuring the activity or the signal of the reporter gene; and/or
d) a cell line expressing the NOD2 receptor and a reporter gene, the transcription of which is under the direct control of the NOD2 signaling pathways, the pro-inflammatory molecules being detected or assayed by measuring the activity or the signal of the reporter gene; and
e) a control line not transfected with an immunity receptor.

35. The method of claim 27, wherein the pro-inflammatory molecules are molecules of bacterial origin.

36. The method of claim 27, wherein the production or purification step or steps is or are chosen from steps of heat treatment, of acidification, of passing over activated carbon, of passing over adsorption resins, of ultrafiltration, of filtration, or of chemical or enzymatic hydrolysis, or combinations thereof.

37. The method of claim 27, wherein the glucose polymers are selected from icodextrin and branched or unbranched maltodextrins, and the glucose polymer hydrolysates are a product of total hydrolysis.

38. The method of claim 27, wherein the samples of glucose polymers or of hydrolysates thereof are prefiltered with a cut-off threshold at 30 kDa, and the filtrate is brought into contact with the cell line used in the test.

39. The method of claim 35, wherein said pro-inflammatory molecules of bacterial origin are PGNs, LPSs, lipopeptides, PGN depolymerization products MDP, formylated microbial peptides or f-MLP or β-glucans.
